(19) United States
(12) Patent Application Publication
(10) Pub. No.: US 2007/0009377 A1
(43) Pub. Date: Jan. 11, 2007

(54) Methods for Reducing Pathogens in Biological Samples

(75) Inventors: Raymond P. Goodrich, Lakewood, CO (US); Cynthia A. Scott, Lakewood, CO (US)

Correspondence Address: GREENLEE WINNER AND SULLIVAN P C, 4875 PEARL EAST CIRCLE, SUITE 200, BOULDER, CO, 80301, US

(73) Assignee: NAVIGANT BIOTECHNOLOGIES, INC., Lakewood, CO (US)

(21) Appl. No.: 11/428,669

(22) Filed: Jul. 5, 2006

Related U.S. Application Data
(60) Provisional application No. 60/696,932, filed on Jul. 6, 2005.

(51) Int. Cl. A01N 1/02; A61L 2/10

(52) U.S. Cl. 422/24; 435/2

(57) ABSTRACT

This invention provides methods, devices and device components for treating biological samples with electromagnetic radiation. The methods, devices and device components of the present invention are capable of providing well characterized, uniform and reproducible net radiant energies and/or radiant powers to biological samples undergoing processing. In addition, the present methods, devices and device components are capable of delivering electromagnetic radiation to biological samples having a distribution of wavelengths selected to provide enhanced pathogen reduction, while minimizing photoinduced damage to components comprising therapeutic and/or reinfusion agents.

### CROSS-REFERENCE TO RELATED APPLICATIONS

This application claims priority under 35 U.S.C. 119(e) to U.S. provisional Patent Application 60/696,932 filed Jul. 6, 2005, which is hereby incorporated by reference in its entirety to the extent not inconsistent with the disclosure herein.

### BACKGROUND OF THE INVENTION

Collection, processing and purification of biological samples are important processes in a range of medical therapies and procedures. Important biological samples used as therapeutic agents include whole blood and purified blood components, such as red blood cells, platelets, white blood cells and plasma. In the field of transfusion medicine, one or more whole blood components are directly introduced into a patient's blood stream to replace a depleted or deficient component. Infusion of plasma-derived materials, such as blood proteins, also plays a critical role in a number of therapeutic applications. For example, plasma-derived immunoglobulin is commonly provided to supplement a patient's compromised immune system. Due to increases in the demand for purified biological samples for transfusion, infusion and transplantation therapies, substantial research efforts are currently directed at improving the availability, safety and purity of biological samples used as therapeutic agents.

The safety and efficacy of transfusion, infusion and transplantation therapies depends on identifying the presence of and/or reducing the biological activities of pathogenic biological contaminants, such as viruses, bacteria, fungi, bacteriophages and protozoa, present in donated biological samples. The presence of pathogens in samples used as therapeutic agents is dangerous as these contaminants a capable of causing infection of patients undergoing treatment and can deleteriously affect recovery time, quality of life and future health. Further, the presence of pathogenic contaminants in biological samples is of serious consequence not only to patients undergoing therapeutic transfusion, infusion and transplantation procedures, but also to doctors and other hospital personnel who routinely handle, process and administer these materials.

While biological samples used as therapeutic agents are currently safer than in the past, the risk of exposure to pathogens in human blood samples remains significant. A large number of deleterious contaminants are routinely identified in intracellular and extracellular fractions of human blood. For example, it is estimated that approximately 1 in 200 thousand donated blood and blood component samples are contaminated with hepatitis B, approximately 1 in 1.9 million are contaminated with human immunodeficiency virus type I/II (HIV), and approximately 1 in 1.6 million are contaminated with hepatitis C. Bacterial contaminants are even more common than viral contaminants in donated blood and blood component samples, and may reach an incidence of contamination in platelet products as high as about 1 in 2000 to 3000 samples. Contamination of donated blood components with donor leukocytes is another frequently encountered problem.

In addition to these known risks, it has also been demonstrated that human blood reservoirs are routinely contaminated with other pathogens which are not assayed in conventional blood screening protocols, including transfusion-transmitted virus, hepatitis E virus, human herpes virus 8, HTLV-2, West Nile virus, hepatitis A, TT virus, SEN-V malaria, babesia, trypanosome, and parvo B19 virus. As a result of the risks associated with these contaminants, whole blood and blood components may currently be underutilized as therapeutic agents, due to concerns of disease transmission.

Over the last decade, a number of methods have emerged for reducing risks associated with pathogenic contaminants in biological samples, especially donated blood components. Screening of donors and acquired blood samples has been demonstrated to provide an effective method for identifying and avoiding pathogen-contaminated biological samples. Effective screening methods combine rigorous donor interviews and pathogen specific assay techniques. Despite reductions in pathogen transmission achieved by screening, these methods remain susceptible to problems associated with the presence of pathogenic contaminants. First, a measurable incidence of pathogen transmission is associated with screened blood samples due to the difficulty of detecting pathogens at very low levels which are capable of causing infection. Second, blood sample screening results in the disposal of large quantities of donated blood that are deemed unusable. As the supply of donated blood is limited, disposal of contaminated blood significantly reduces the availability of blood components needed for important therapeutic procedures. Third, current screening methodologies are limited to approximately nine pathogen-specific assays. Accordingly, a number of pathogens known to be present in blood samples are not currently assayed, not to mention those blood pathogens present in human blood which have yet to be identified. Finally, screening methods are costly and labor intensive, requiring the expenditure of a great deal of resources to be implemented effectively.

A different approach to reducing the risks associated with contamination of biological samples involves decreasing the biological activities of pathogens present in biological samples by killing the pathogens or rendering them incapable of replication. Over the last decade, a variety of methods for reducing the biological activities of pathogens in biological fluids have emerged including direct photoreduction, the use of detergents for inactivating viruses having lipid membranes, chemical treatment methods and photoinduced chemical reduction techniques. Due to its compatibility with high-volume pathogen inactivation and demonstrated efficacy, photoinduced chemical reduction and direct photoreduction are two especially promising techniques for treating biological samples. U.S. Pat. Nos. 6,277,337, 5,607,924, 5,545,516, 4,915,683, 5,516,629, and 5,587,490 describe systems and methods for photoinduced chemical reduction and direct photoreduction for inactivating pathogens in blood.

In photoinduced chemical reduction methods, effective amounts of one or more photosensitizers are added to a biological fluid, which is subsequently mixed continuously and illuminated with electromagnetic radiation. Illumination activates the photosensitizers, thereby initiating chemical reactions and/or physical processes which kill the pathogens present in the sample or substantially prevent pathogens from replicating. In direct photoreduction methods, a biological sample is illuminated with electromagnetic radiation having wavelengths that directly provide pathogen destruction or inactivation. Photoinduced chemical reduction methods are preferred to direct photoreduction in some pathogen reduction applications because these techniques are often compatible with illumination wavelengths, radiant intensities and radiant energies which do not significantly affect the biological activities and viabilities of therapeutic components of a biological fluid undergoing treatment.

Effective photoinduced chemical reduction of pathogens in biological fluids requires achieving and maintaining effective illumination and fluid mixing conditions during sample treatment. First, the wavelength distribution of the activating electromagnetic radiation must be within the absorption range of the photosensitizer(s) present, preferably centered close to absorbance maxima. Second, illumination intensities and radiant energies provided to all portions of the fluid undergoing pathogen reduction must be sufficient to excite a population of photosensitive reagents in the sample that is large enough to reduce the biological activities of pathogens to a desired level. Finally, fluid mixing rates must be sufficiently large to evenly distribute the photosensitizers and radiant energies throughout the entire volume of the fluid undergoing treatment.

Despite the demonstrated efficacy of photoinduced chemical reduction and direct photoreduction, the full benefits of these techniques for reducing the biological activities of pathogens in blood and blood components are currently hindered due to problems arising from the optical properties of conventional containers for holding biological samples during treatment. First, the amount of electromagnetic radiation delivered to a sample depends on the transmission properties of the container in which it is held during treatment. However, many materials used in conventional blood bags and containers, such as poly(vinyl chloride) materials having di-2-ethylhexyl phthalate (DEHP) plasticizers, are known to undergo photochemically induced chemical and/or physical changes upon exposure to ultraviolet and visible electromagnetic radiation. These changes are capable of significantly affecting the transmission properties of these materials. These unwanted photochemical processes are also very difficult to characterize as a function of exposure time and seriously undermine efforts to quantify the amount of radiation actually delivered to a sample during a specific treatment protocol. Variations in the transmission properties of containers for biologic samples during a pathogen reduction treatment process obscure accurate determination of the extent of pathogen reduction achieved, undermine quality control efforts and may negatively impact product validation and regulatory approval. Second, many conventional containers for biological samples, such as polyolefin bags, are at least partially transparent to high energy, ultraviolet electromagnetic radiation that degrade the viability and biological activity of healthy cells, tissues and biological molecules, such as proteins. As many conventional optical sources used in processing biological samples, such as arc discharge lamps, mercury vapor fluorescent lamps, cold cathode fluorescent lamps and excimer lamps, generate significant amounts of high energy, ultraviolet electromagnetic radiation, components of biological samples undergoing pathogen reduction often undergo unwanted photoinduced degradation at least to some extent during processing.

It will be appreciated from the foregoing that a clear need exists for methods and devices for treating biological fluids with electromagnetic radiation that ensure their safe and effective use as therapeutic agents. Specifically, methods, devices and device components are needed that ensure reproducible and well characterized radiant energies are provided to biological samples undergoing direct and/or photoinduced chemical reduction of pathogens. In addition, methods, devices and device components are needed which avoid or minimize exposure of components of a biological sample comprising therapeutic agents to electromagnetic radiation capable of deleteriously affecting their biological activities and viabilities.

### SUMMARY OF THE INVENTION

This invention provides methods, devices and device components for treating samples with electromagnetic radiation. The present invention provides methods and systems for reducing the biological activities of pathogens in biological samples providing improved pathogen reduction effectiveness relative to conventional pathogen reduction treatment processes, and which optimize the biological activities and viabilities of therapeutic and reinfusion agents derived from treated biological samples.

It is an object of the present invention to provide methods and devices for treating biological samples so that they are safe and effective for use as a therapeutic agent or reinfusion agent. It is further an object of the present invention to provide methods and devices capable of providing reproducible and uniform net radiant energies and/or radiant powers to biological samples undergoing treatment with electromagnetic radiation. It is further an object of the present invention to provide methods and devices for treating biological samples with electromagnetic radiation having radiant powers and net radiant energies that are capable of being accurately quantified, calculated and/or predicted.

In one aspect, the present invention provides methods for reducing pathogens in a biological sample wherein the sample is provided in a container having optical properties, such as extinction coefficients, absorption cross sections, and percentages of transmission, that are substantially constant during exposure of the container to electromagnetic radiation throughout a treatment process. In the context of this description, “substantially constant” extinction coefficients, absorption cross sections, and percentages of transmission change by less than about 10% over a given treatment process, preferably less than about 5% for some applications. In one embodiment of this aspect of the present invention, a biological sample, such as blood or a blood component, is provided in a container comprising a polymeric material and at least one additive such as a plasticizer, wherein the combination of the polymeric material and additive(s) comprising the container are capable of at least partially transmitting electromagnetic radiation having a selected distribution of wavelengths, for example a distribution of wavelengths providing direct photoreduction of pathogens in the biological sample and/or a distribution of wavelengths that are capable of inducing photochemical reactions resulting in pathogen reduction.

In this aspect of the present invention, the container having the biological sample is exposed to electromagnetic radiation, such as electromagnetic radiation having wavelengths in the visible and/or ultraviolet regions of the electromagnetic spectrum. Electromagnetic radiation having the selected distribution of wavelengths is at least partially transmitted by the container, and interacts with the biological sample (and/or additives provided therein) held in the container, thereby reducing pathogens present in the sample. In this aspect of the present invention, the physical, chemical and optical properties the combination of polymer material and additive(s) comprising the container are selected such that the transmission of electromagnetic radiation having the selected distribution of wavelengths by the container is substantially constant during the entire processing protocol (i.e. the exposure period to electromagnetic radiation) for a given treatment procedure. Substantially constant transmission characteristics of containers of this aspect of the present invention are provided by selection of a combination of polymer material(s) and additives(s) that do not undergo significant photoinduced changes in their extinction coefficients (or alternatively percentages of transmission) for light having the selected distribution of wavelengths upon exposure to ultraviolet and/or visible electromagnetic radiation.

Methods of this aspect of the present invention may further comprise the steps of measuring or otherwise characterizing optical properties of the container, such as the percentages of transmission and/or extinction coefficients prior to processing of the biological sample, and continuously, periodically or intermittently monitoring the radiant power of electromagnetic radiation provided to the container during treatment of the biological sample. In this aspect of the present invention the percentages of transmission (or alternatively extinction coefficients) of the container are characterized as a function of wavelength prior to treatment and used in combination with the measured radiant power, radiant energy or both of an optical source to determine and/or control the radiant energies and/or radiant powers provided to the biological sample during treatment. Use of a source of electromagnetic radiation providing a substantially constant radiant power allows the exposure time required to achieve a selected extent of pathogen reduction to be accurately predicted, calculated and/or controlled.

A significant advantage of methods of the present invention employing containers comprising a combination of a polymeric material and one or more additives exhibiting optical properties, such as extinction coefficients and percentage transmittances corresponding to the first distribution of wavelengths, that are substantially constant during exposure to electromagnetic radiation is that these methods allow for accurate characterization and/or measurement of net energies actually delivered to sample during processing. This feature of the present invention is beneficial for avoiding exposure of biological samples to net radiant energies insufficient to achieve a selected extent of pathogen reduction and useful for avoiding overexposure of a biological sample to net radiant energies and/or radiant powers greater than those needed to achieve a selected extent of pathogen reduction, for example avoiding exposure of a sample to radiant powers and/or radiant energies resulting in damage and/or degradation of components of the biological sample comprising therapeutic agents.

Useful polymeric materials and additives for containers of this aspect of the present invention do not exhibit significant changes (i.e. less than about 10% or more preferably for some applications less than about 5%) in percentages of transmission and/or extinction coefficients for electromagnetic radiation of the first distribution of wavelengths upon exposure to radiant powers, net radiant energies, and incident wavelengths and for exposure times useful for reducing pathogens in biological samples, such as blood and blood components. Useful materials comprises a combination of polymeric materials and additives which exhibit good photolytic stability and are, thus, resistant to changes in chemical composition and/or physical state induced by the absorption of electromagnetic radiation, particularly ultraviolet and visible electromagnetic radiation. This important functionality is achieved by appropriate selection of the compositions, physical states, conjugation scheme and concentrations of polymeric materials and additives comprising containers useful in the methods of the present invention, and represents a significant improvement over conventional containers for biological samples, such as those comprising poly(vinyl chloride) materials having DEHP plasticizers, which undergo significant photochemically induced changes upon absorption of ultraviolet radiation that decrease the ability of these materials to transmit electromagnetic radiation useful for pathogen reduction.

In an exemplary embodiment, polymeric materials and additives comprising containers of this aspect of the present invention exhibit a less than about 10 % change in percentages of transmission and/or extinction coefficients for electromagnetic radiation of the first distribution upon exposure to net radiant energies selected over the range of about 0.1 J cm⁻² to about 24 J cm⁻² using exposure times as large as 30 minutes.

In an embodiment of this aspect of the present invention, poly(vinyl chloride) in combination with one or more citrate plasticizers, such as n-butyryltri-n-hexyl citrate, triethyl citrate, acetyltriethyl citrate; and acetyltri-n-butyl citrate, provide materials for containers having optical, mechanical and toxological properties useful for treating blood and blood component samples with electromagnetic radiation. First, poly(vinyl chloride) in combination with one or more citrate plasticizers provide materials for containers that effectively transmit electromagnetic radiation having wavelengths ranging from about 285 nanometers to about 500 nanometers corresponding to electromagnetic radiation useful for direct photoreduction and/or photoinduced chemical reduction methods. Electromagnetic radiation having this range of wavelengths is efficiently absorbed by some photosensitizers, such as 7,8-dimethyl-10-ribityl isoalloxazine (in bound or unbound states in a biological sample). Second, poly(vinyl chloride) in combination with one or more citrate plasticizers provide materials for containers that do not undergo significant changes in percentages transmission and extinction coefficients upon exposure to electromagnetic radiation having wavelengths useful for blood processing. For example, use of poly(vinyl chloride) in combination with n-butyryltri-n-hexyl citrate (having a concentration of about 38% by weight) provides containers that exhibit a less than 10% change in the percentages of transmission corresponding to electromagnetic radiation having wavelengths over the range of about 285 nanometers to about 365 nanometers during treatment of a blood or blood components. Third, poly(vinyl chloride) in combination with one or more citrate plasticizers provide containers that are permeable with respect to oxygen (O₂) and carbon dioxide (CO₂) gases, which is beneficial for storing certain blood products and blood components without damaging these materials, such as platelet containing blood components and products. Furthermore, the permeability of containers comprising poly(vinyl chloride) in combination with one or more citrate plasticizer with respect to oxygen and carbon dioxide does not decrease significantly after exposure to electromagnetic radiation useful for treating blood and blood components. This aspect also allows blood and blood components containing platelets to be stored in the same container used during a pathogen reduction treatment process, thereby avoiding an extra sample transfer step after photoprocessing to permeable storage container. Finally, poly(vinyl chloride) in combination with one or more citrate plasticizers are nontoxic materials, and therefore, containers made of these materials do not release toxic agents to a biological sample during treatment with electromagnetic radiation or during storage subsequent to treatment. Accordingly, biological samples, such as blood and blood components, processed and stored in container comprising poly(vinyl chloride) in combination with one or more citrate plasticizers may be safely administered to patients as therapeutic agents and/or reinfusion agents.

In another aspect, the present invention provides methods for reducing pathogens in a biological sample wherein a biological sample undergoing treatment is provided within a container that serves as an optical component for filtering incident electromagnetic radiation, in addition to holding the biological sample during treatment. In this aspect of the present invention, the container comprises an integrated optical filtering element. In one embodiment, for example, the container comprises one or more materials that are capable of absorbing and/or scattering a portion of the incident electromagnetic radiation, thereby at least partially preventing certain wavelengths of light from interacting with the biological sample undergoing treatment.

In one embodiment of this aspect of the present invention, a method for reducing pathogens in a biological sample comprises the step of providing a container holding the biological sample, wherein the container comprises a polymeric material and at least one optical filtering additive, such as an additive immobilized within the polymer network, capable of absorbing and/or scattering undesirable electromagnetic radiation, such as electromagnetic radiation capable of damaging or degrading the sample. In this embodiment of the present invention, the composition and concentration of the additive(s) and thickness of the container are selected so that electromagnetic radiation having a first distribution of wavelengths is transmitted by the container, while transmission of electromagnetic radiation having a second distribution of wavelengths is substantially prevented. In the context of this description, the expression the “transmission of electromagnetic radiation having a second distribution of wavelengths is substantially prevented” refers to percentages of transmission less than about 10% and less than about 5% for some applications. In an embodiment useful for reducing pathogens in blood and blood components, the first distribution of wavelengths corresponds to electromagnetic radiation capable of initiating pathogen reduction directly and/or via initiating photochemical reactions involving one or more photosensitizers, and the second distribution of wavelengths corresponds to electromagnetic radiation capable of damaging or degrading beneficial components of the biological sample, such as cells, proteins and organelles. This method further comprises the step of exposing the container to electromagnetic radiation and, as a result of the optical properties of the additive(s) comprising the container, transmission of electromagnetic radiation having the second distribution of wavelengths is substantially prevented. In contrast, electromagnetic radiation having the first distribution of wavelengths is transmitted by the container and interacts with components of the biological sample, thereby reducing the pathogens in the biological sample. Accordingly, the container used in this aspect of the present invention itself functions as an optical filter allowing the transmission of electromagnetic radiation useful for initiating pathogen reduction while minimizing transmission of electromagnetic radiation capable of damaging components of the biological sample, such as components comprising therapeutic and/or reinfusion agents.

In this aspect of the present invention, selection of the composition and concentration of additives comprising the container, at least in part, determines the optical transmission properties of the container, such as which wavelengths of light are transmitted, absorbed and/or scattered. Useful containers in this aspect of the invention comprise additives that transmit electromagnetic radiation having wavelengths capable of directly or indirectly initiating pathogen reduction, such as light having wavelengths between about 285 nanometers and about 550 nanometers, and that substantially prevent transmission of electromagnetic radiation having wavelengths that degrade the viability and/or biological activity of components of the biological sample comprising therapeutic and/or reinfusion agents, such as light having wavelengths less than about 285 nanometers.

In an exemplary embodiment useful for pathogen reduction in blood or blood component samples containing platelets, additives for optical filtering applications are nontoxic, do not substantially reduce the permeability of the container for platelet storage with respect to CO₂ and O₂ and do not negatively affect beneficial mechanical properties (e.g. strength, flexibility and durability) of the container. Useful additives in the methods of the present invention providing optical filtering functionality include amino acids such as tyrosine, histidine, phenylalanine and tryptophan, peptides and/or proteins that absorb light having wavelengths over the wavelength range of about 200 nanometers to about 270 nanometers. Amino acid, peptides and protein additives may be provided as polymer components of a copolymer wherein they are covalently linked to other polymer materials in the network of a copolymer. Alternatively, amino acid, peptide and protein additives may be provided as additive materials dispersed and immobilized in a polymer network but not necessarily covalently bonded to the network. Use of amino acid, peptide and/or protein additives in this aspect of the present invention is particularly useful for protecting against photoinduced degradation of blood and blood component samples, because the absorption spectra of these additives overlap significantly with the spectra of many proteins in these samples, and thus the amino acid, peptides and/or protein additives in the container substantially prevent transmission of light that would otherwise be absorbed by proteins present in the sample. Useful additives also include nucleic acids and/or oligonucelotides immobilized in a polymer network either in the form of a copolymer or a dispersed phase, and include synthetic and naturally occurring pigments and dyes.

A wide variety of polymeric materials are useful in the methods of the present invention including, but not limited to, thermoplastics, thermosets reinforced plastics and composite polymeric materials. In addition, a wide variety of additives are useful in the methods of the present invention including, but not limited to, plasticizers, light stabilizers, heat stabilizers, antioxidants, flame retardants, release agents, nucleating agents, pigments and other optical absorbers. Containers of the present invention may further comprise other materials such as fibers, particulate materials and other structural enhancers.

The concentration of additives in containers of the present invention establishes, at least in part, the optical transmission properties of containers for biological samples. The larger the concentration of additive, such as optical absorbers, pigments and citrate plasticizers, the greater the extent of optical filtering provided by the container. In addition, the concentration of additive may affect the photolytic stability of the container (i.e. the ability to provide substantially constant transmission properties during exposure to electromagnetic radiation). In an embodiment of the present invention useful for both direct photoreduction and photoinduced chemical reduction of pathogens in blood and blood component samples, the concentration of citrate plasticizers in poly(vinyl chloride) is selected over the range of about 25% to about 50% by mass, preferably about 38% by weight for some applications.

The present methods are particularly useful for reducing pathogens in blood components including, but not limited to, platelet-containing and/or plasma-containing blood components. Exemplary methods of treating platelet and/or plasma containing blood components involve exposure of these materials to electromagnetic radiation having a distribution of wavelengths selected over the range of about 285 nm to about 365 nm. Optionally, methods of this aspect of the present invention may further comprise the step of adding one or more sample additives to the biological sample in the container, such as photosensitizers, enhancers, stabilizing agents, preservatives, dilutants or anticoagulation agents. In an embodiment of the present invention comprising a method of photoinduced chemical reduction of pathogens, 7,8-dimethyl-10-ribityl isoalloxazine is provided to a platelet-containing and/or plasma-containing blood component prior to exposure to electromagnetic radiation.

Containers useful in the present methods may have any volume, size, shape and surface area useful for processing biological samples. Containers of the present invention included fluid containers, such as bags, flexible containers, collapsible containers, tubes, reaction vessels, chambers, buckets, troughs and all equivalents of these known in the art of processing biological materials. Containers useful in methods of the present invention may be entirely fabricated from polymeric materials and additives. Alternatively, the present methods are compatible with containers having discrete partially transparent regions comprising polymeric materials and additives. Containers of the present invention may have a plurality of partially transparent regions allowing for illumination via exposure of a plurality of surfaces of the container to electromagnetic radiation.

Containers useful in the present methods may be provided with identifying indicia, such as a bar code, written label or area for handwritten notations. Optionally, containers useful in the present methods may be operably connect to a fluid mixing means, such as an agitator, mixer, fluid pump, recirculator or stirrer, for mixing a biological sample comprising a fluid during processing. Optionally, containers useful in the present methods may be configured in a manner such that they may be integrated into a blood processing apparatus, such as a density centrifuge, elutriation chamber, photoreactor, washing chamber and the COBE® Spectra™ or TRIMA® apheresis systems, available from Gambro® BCT®, Lakewood, Colo., USA. The methods of the present invention are suited for the treatment of fluids, particularly biological fluids, contained in an at least partially transparent fixed-volume container. In this context the term fixed volume container refers to a closed space, which may be made of a rigid or flexible material. The methods and devices of the present invention are also applicable to treatment of fluids, particularly biological fluids, flowing through a container comprising a flow reactor. In one embodiment, fluid is flowed through the flow reactor at a flow velocity selected to establish a residence time of the fluid in the illuminated portion(s) of the flow reactor providing a desired extent of reduction in the biological activities of pathogens present. Fluid flow conditions in the flow reactor may have a laminar component, a turbulent component or a mixture of both laminar and turbulent components.

The methods of the present invention are also useful for reducing the biological activities of leukocytes present in a biological sample, such as blood or component(s) thereof. Reducing the biological activity of leukocytes, commonly referred to as leukoreduction, is often desirable when suppression of immune responses or autoimmune responses is desired for the administration of a therapeutic agent derived from blood. For example, reduction of leukocyte biological activity may be beneficial in processes involving transfusion of red blood cells, platelets and/or plasma when patient or donor leukocytes are present. In exemplary embodiments, a biological sample undergoing a leukoreduction treatment is provided in a container having optical transmission properties that are substantially constant during a period of exposure to electromagnetic radiation in a selected treatment procedure. The present invention also includes methods wherein a biological sample is held in a container providing optical filtering that minimizes the exposure of components of the sample to harmful high energy ultraviolet electromagnetic radiation, while providing exposure to electromagnetic radiation capable of reducing the biological activities of leukocytes present in the sample.

The methods and device of the present invention are broadly applicable to any process whereby a biological sample is exposed to electromagnetic radiation. In one embodiment, the present methods comprise methods of reducing the biological activities of pathogens in blood or blood components, such as red blood cell-containing blood components, platelet containing blood components, plasma containing components, white blood cell containing components and solutions containing one or more proteins derived from blood, which provide an improved blood product quality over conventional pathogen reduction methods. In another embodiment, the present invention provides methods of reducing the biological activities of pathogens in fluids which are administered as therapeutic agents, such as intravenous medicines or peritoneal solutions.

In another aspect the present invention provides a method for reducing pathogens in a biological sample comprising the steps of: (1) providing a container holding the biological sample; wherein the container comprises a polymeric material and at least one additive, and wherein the container transmits electromagnetic radiation having a distribution of wavelengths; and (2) exposing the container to electromagnetic radiation, wherein electromagnetic radiation having the distribution of wavelengths is transmitted by the container and is at least partially absorbed by the biological sample, thereby reducing the pathogens in the biological sample; wherein the transmission of electromagnetic radiation having the distribution of wavelengths by the container is substantially constant during exposure to electromagnetic radiation. In an embodiment, the additive is one or more citrate plasticizers, such as n-butyryltri-n-hexyl citrate, triethyl citrate, acetyltriethyl citrate; and acetyltri-n-butyl citrate.

In another aspect the present invention provides a method for reducing pathogens in a biological sample comprising the steps of: (1) providing a container holding the biological sample; wherein the container comprises a polymeric material and at least one additive, wherein the composition and concentration of the additive is selected so that electromagnetic radiation having a first distribution of wavelengths is transmitted by the container and transmission of electromagnetic radiation having a second distribution of wavelengths is substantially prevented, wherein electromagnetic radiation having the first distribution of wavelengths is capable of initiating pathogen reduction of the biological sample and wherein electromagnetic radiation having the second distribution of wavelengths is capable of damaging the biological sample; and (2) exposing the container to electromagnetic radiation, wherein transmission of electromagnetic radiation of the second distribution of wavelengths is substantially prevented, and wherein electromagnetic radiation having the first distribution of wavelengths is transmitted by the container and is at least partially absorbed by the biological sample, thereby reducing the pathogens in the biological sample. In an embodiment, the additive is one or more citrate plasticizers, such as n-butyryltri-n-hexyl citrate, triethyl citrate, acetyltriethyl citrate; and acetyltri-n-butyl citrate. In an embodiment, the additive is one or more amino acids such as tyrosine, histidine, phenylalanine and tryptophan, or peptides and/or proteins containing these amino acids.

### BRIEF DESCRIPTION OF THE DRAWINGS

FIG. 1 shows a schematic diagram illustrating a method of reducing pathogens in blood or a component thereof held in a container comprising poly(vinyl chloride) and a citrate plasticizer.

FIG. 2 provides a schematic diagram of an exemplary container comprising an at least partially transparent bag for holding a blood or blood component sample.

FIG. 3 shows an absorption spectrum of a 200 micromolar solution of 7,8-dimethyl-10-ribityl isoalloxazine in phosphate buffer saline (curve A) which is characterized by absorption maxima at about 370 nanometers, 450 nanometers, 260 nanometers and 220 nanometers. FIG. 3 also shows an action spectrum (log virus kill; curve B) corresponding to the reduction efficiency of a platelet-containing sample having 7,8-dimethyl-10-ribityl isoalloxazine and exposed to selected wavelengths of ultraviolet and visible electromagnetic radiation.

FIG. 4 shows transmission spectra of a container useful in the present methods comprising a poly(vinyl chloride) and citrate plasticizer bag (curve A) and a container comprising a conventional polyolefin bag (curve B).

FIG. 5A shows transmission spectra of a citrate plasticized poly(vinyl chloride) bag upon successive exposures to ultraviolet radiation and FIG. 5B provides a plot of the percentage transmission at 308 nanometers as a function of exposure time.

FIG. 6A shows transmission spectra of a poly(vinyl chloride) and DEHP plasticizer bag upon successive exposures to ultraviolet radiation and FIG. 6B provides a plot of the percentage transmission of this bag at 308 nanometers as a function of exposure time.

FIG. 7A shows transmission spectra of a polyolefin bag upon successive exposures to ultraviolet radiation and FIG. 7B provides a plot of the percentage transmission of this bag at 308 nanometers as a function of exposure time.

FIGS. 8A-H shows correlations of in vitro cell quality parameters with in vivo platelet recovery. The in vivo platelet recovery is function of values of lactate production (a), pH at 22° C. on day 5 (b), glucose consumption (c), P-selectin expression percent on day 5 (d), swirl score on day 5 (e), HSR percent on day 5 (f), pO₂ (g) and pCO₂ on day 5 (h). In the graphs provided in FIGS. 8A-8H the open circles correspond to control platelets, solid diamonds correspond to medium dose of UV light treated platelets and solid squares correspond to high dose of UV light treated platelets.

FIG. 9 shows measured O₂ transmission rates for each sample (three bag samples for test and control groups, two replicates per sample).

FIG. 10 shows the mean for each group (test and control) with error bars indicating ±1 standard deviation.

FIG. 11 shows measured CO₂ transmission rates for each sample (three bag samples for each group, two replicates per sample).

FIG. 12 shows the mean of CO₂ transmission rates for each group (test and control) with error bars indicating ±1 standard deviation.

Referring to the drawings, like numerals indicate like elements and the same number appearing in more than one drawing refers to the same element. In addition, hereinafter, the following definitions apply:

“Citrate plasticizer” refers to a citrate ester, such as an alcohol ester of citric acid, which is added to a polymeric material, such as poly(vinyl chloride) to provided desired mechanical, physical, chemical and optical properties, including enhanced flexibility, softness, extensibility, impact resistance or any combination of these. Citrate plasticizers useful in methods and devices for treating biological samples comprising therapeutic agents are nontoxic. Exemplary citrate plasticizers include, but are not limited to, n-butyryltri-n-hexyl citrate, triethyl citrate, acetyltriethyl citrate, tri-n-butyl citrate; and acetyltri-n-butyl citrate.

The terms “electromagnetic radiation” and “light” are used synonymously in the present description and refer to waves of electric and magnetic fields. Electromagnetic radiation useful for the methods of the present invention includes, but is not limited to, ultraviolet light, visible light, or any combination of these. Selection of the wavelength distribution of electromagnetic radiation used in the methods of the present invention may be based on a number of factors including, but not limited to, the absorption spectrum of one or more photosensitive materials provided to a biological sample undergoing treatment, the transmission, absorption and/or scattering coefficients of components of the biological sample as a function of wavelength, the wavelengths of electromagnetic radiation which is harmful to components of a biological sample or any combination of these. Exemplary methods use electromagnetic radiation characterized by a distribution of wavelengths that are substantially absorbed by photosensitive materials provided to the fluid and are substantially transmitted by the fluid itself within at least a portion of the fluid. Exemplary methods and devices of the present invention useful for treating red blood cell-containing blood components use electromagnetic radiation having wavelengths in the visible region of the electromagnetic spectrum. For example, in one aspect of the present invention useful for treating red blood cell-containing blood components and employing a photosensitive material which absorbs light in the visible region of the electromagnetic spectrum, electromagnetic radiation having a distribution of wavelengths selected over the range of about 400 nm to about 800 nm is employed. Exemplary methods and devices of the present invention useful for treating plasma and platelet-containing blood components use electromagnetic radiation having wavelengths in the ultraviolet region of the electromagnetic spectrum. For example, in one aspect of the present invention which may be useful for treating platelet and plasma-containing blood components and employing a photosensitive material comprising 7,8-dimethyl-10-ribityl isoalloxazine, electromagnetic radiation having a distribution of wavelengths selected over the range of about 285 nm to about 365 nm is employed. As will be understood by persons skilled in the art, the absorption spectrum of photosensitive materials, such as 7,8-dimethyl-10-ribityl isoalloxazine, may vary when in the presence of certain fluid components, such as proteins, and the present methods may take this change in the absorption spectrum of photosensitive material in to account in the selection of the appropriate distribution of wavelengths of electromagnetic radiation provided to biological samples having photosensitive materials.

“Net radiant energy” refers to the total amount of radiant energy delivered to a fluid during a fluid treatment process or combination of fluid treatment processes. Net radiant energy may be expressed in terms of power, exposure time and illuminated surface area by the equation;

$${E}_{net} = {\int }_{A = 0}^{A = {A}_{I}} {\int }_{t = 0}^{t = {t}_{f}} P ( t , A ) ⅆ A ⅆ t ; \qquad ( I )$$

wherein Eₙₑₜ is the net radiant energy delivered, P(t) is the power of the electromagnetic radiation exposed to the fluid as a function of time and area, t_{f} is the time interval for illumination, t is time, A is area and Aₗ is the illuminated area of the container holding the fluid. In methods of the present invention employing a substantially constant power, net radiant energy may be expressed in terms of radiant power and exposure time by the equation:
Eₙₑₜ=P×t_{f}; (II)
wherein Eₙₑₜ is the net radiant energy, P is the constant radiant power of the electromagnetic radiation and t_{f} is the time interval for illumination. Net radiant energy may also be expressed per unit area or per unit volume.

“Treating” or “processing” a biological sample with electromagnetic radiation refers to a process whereby electromagnetic radiation is delivered to a biological sample to achieve a desired change in the composition of the biological sample or components of the biological sample and/or to achieve a change in the biological activities of one or more components of the biological sample. In one aspect, the methods of the present invention are capable of treating a biological sample, including biological fluids such as blood, and components of blood, with electromagnetic radiation in such a manner as to reduce the biological activities of one or more pathogens present in the biological sample. In another aspect, the methods of the present invention are capable of treating a biological sample with electromagnetic radiation in such a manner as to reduce the biological activities of one or more leukocytes present in the biological sample.

The terms “intensity” and “intensities” refers to the square of the amplitude of an electromagnetic wave or plurality of electromagnetic waves. The term amplitude in this context refers to the magnitude of an oscillation of an electromagnetic wave. Alternatively, the terms “intensity” and “intensities” may refer to the time average energy flux of a beam of electromagnetic radiation or plurality of beams of electromagnetic radiation, for example the number of photons per square centimeter per unit time of a beam of electromagnetic radiation or plurality of beams of electromagnetic radiation.

“Component of a biological sample” and ‘biological sample component” are used synonymously in the present description and refer to a portion or fraction of a biological sample. Components of a biological sample may include particles, molecules, ions, cells and fragments of cells, photosensitizers, pathogens, aggregates of molecules and complexes, aggregates of pathogens, leukocytes or any combinations of these.

“Photosensitizers” refer to materials that absorb electromagnetic radiation and utilize the absorbed energy to carry out a desired chemical or physical process. Photosensitizers for blood treatment applications are capable of initiating a reduction in the biological activities of pathogens and/or leukocytes present in a biological sample upon absorption of electromagnetic radiation. Photosensitizers useful for some applications of the present invention include compounds that preferentially bind, absorb or intercalate to nucleic acids, thereby focusing their photodynamic effects upon microorganisms, virus and leukocytes. Exemplary photosensitizers which may be useful in the methods of the present invention include, but are not limited to, alloxazine compounds, isoalloxazine compounds, 7,8-dimethyl-10-ribityl isoalloxazine, porphyrins, psoralens, dyes such as neutral red, methylene blue, acridine, toluidines, flavine (acriflavine hydrochloride) and phenothiazine derivatives, coumarins, quinolones, quinones, and anthroquinones. Photosensitizers useful in the practice of the present invention include nontoxic, endogenous photosensitizers, which do not require removal from a biological sample comprising therapeutic components prior to administration into a patient. Photosensitizers may exist in ionized, partially ionized or neutral states in a biological sample undergoing treatment. Photosensitizers may exist as aggregates of compounds and molecular complexes in a biological sample undergoing treatment.

The term “endogenous” means naturally found in a human or mammalian body, either as a result of synthesis by the body or due to ingestion as an essential foodstuff (e.g. vitamins) or formation of metabolites and/or byproducts in vivo. The term “non-endogenous” means not naturally found in a human or mammalian body, either as a result of synthesis by the body or due to ingestion of an essential foodstuff or formation of metabolites and/or byproducts in vivo.

“Enhancer” refers to materials added to a biological sample undergoing treatment to make the desired treatment process more efficient and selective. Enhancers include antioxidants or other agents added to prevent degradation of biological sample components comprising therapeutic agents. In addition, enhancers include materials which improve the rate of reduction of the biological activities of pathogens and/or leukocytes. Exemplary enhancers include, but are not limited to, adenine, histidine, cysteine, propyl gallate, glutathione, mercaptopropionylglycine, dithiothreotol, nicotinamide, BHT, BHA, lysine, serine, methionine, gluscose, mannitol, trolox, glycerol and any combination of the compounds.

“Biological sample” broadly refers to any material which is derived from an organism. Biological samples useable with methods of the present invention include, but are not limited to, liquids, and mixtures of more than one liquid, colloids, foams, emulsions, sols, and any combination of these. Biological samples useable in the methods of the present invention include biological fluids, such as whole blood, blood components, blood subcomponents, plasma-containing blood components, platelet-containing blood components, red blood cell-containing blood components, white blood cell-containing blood components, solutions containing one or more proteins derived from blood, or any combinations of these. Exemplary biological samples also include peritoneal solutions used for peritoneal dialysis, intravenous medicines, injectable medicines, nutritional fluids, food stuffs, fermentation media generated from recombination methods, materials produced by recombinant techniques including therapeutic and diagnostic materials, materials produced from transgenic animals and plants including therapeutic and diagnostic materials, milk and milk products, and vaccines. The term biological sample is intended to include samples also comprising one or more sample additives, such as photosensitizes, anticoagulants, stabilizers, enhancers and diluents. Biological samples useful in the methods of the present invention specifically include, but are not limited to, biological samples having one or more photosensitizers present, such as 7,8-dimethyl-10-ribityl isoalloxazine.

“Blood,” “blood product” and “blood component” as used herein include whole blood, blood components and materials which may be derived from whole blood or a component thereof. “Blood,” “blood product” and “blood component” as used herein also include blood, blood components and/or blood products treated with one or more additives, such as an anticoagulant agent, enhancer, photosensitizer, preservative or diluents. “Blood,” “blood product” and “blood component” also refer to mixtures of these materials and additives, such as photosensitizers, enhancers, stabilizers, anticoagulant agents and preservatives. Cellular blood components include, but are not limited to erythrocytes (red blood cells), leukocytes (white blood cells), thrombocytes (platelets), esinophils, monocytes, lymphocytes, granulacytes, basophils, plasma, and blood stems cells. Non-cellular blood components include plasma, and blood proteins isolated from blood samples including, but not limited to, factor III, Von Willebrand factor, factor IX, factor X, factor XI, Hageman factor, prothrombin, anti-thrombin III, fibronectin, plasminogen, plasma protein fraction, immune serum globulin, modified immune globulin, albumin, plasma growth hormone, somatomedin, plasminogen, streptokinase complex, ceruloplasmin, transferrin, haptoglobin, antitrypsin and prekallikrein.

“Non-toxic” is a characteristic of materials that they do not result in a substantially deleterious effects when administered to a patient, person, animal or plant. Non-toxic materials useful for some blood treatment processes are less toxic than porphyrin and porphyrin derivatives and metabolites, which are commonly used for blood sterilization.

“Nucleic acid” includes both ribonucleic acid (RNA) and deoxyribonucleic acid (DNA).

“Partially transparent” refers to the property of a material, device or device component which when illuminated transmit intensities of at least a portion of the incident electromagnetic radiation.

“Pathogen reduction” refers to a process which partially or totally prevents pathogens from reproducing. Pathogen reduction may occur by directly killing pathogens, interfering with their ability to reproduce, or a combination of these processes. Pathogen reduction reduces the biological activities of pathogens present in a fluid. In an exemplary embodiment, the methods and devices of the present invention are capable of reducing the biological activities of pathogens present in a biological fluid such that the fluid is safe for administration as a therapeutic agent.

“Light source” or “source of electromagnetic radiation” refers to any device or material capable of generating electromagnetic radiation or a plurality of devices or materials capable of generating electromagnetic radiation. Exemplary light sources useable in the present invention include, but are not limited to, mercury vapor fluorescent lamps, cold cathode fluorescent lamps, excimer lamps, light emitting diodes (LEDs), arrays of light emitting diodes, arc discharge lamps and tungsten-filament lamps.

“Pathogenic contaminants” and “pathogens” refer to viruses, bacteria, bacteriophages, fungi, protozoa, blood-transmitted parasites. Exemplary viruses include human immunodeficiency virus (HIV), hepatitis A, B, C and G viruses, sindbis virus, cytomegalovirus, vesicular stomatitis virus, herpes simplex viruses, human T-lymphotropic retroviruses, HTLV-III, lymphadenopathy virus LAV/IDAV, parvovirus, transfussion (TT) virus, Epstein-Barr virus, West Nile virus and others known to the art. Exemplary bacteriophages include but are not limited to ΦX174, Φ6, λ, R17, T4 and T2. Exemplary bacteria include P. aeruginosa, S. aureus, S. epidernis, L. monocytogenes, E. coli, K pneumonia and S. marcescens. Exemplary parasites include malaria, babesia and trypanosome.

“Biologically active” refers to the capability of a composition, material, microorganism, or pathogen to effect a change in a living organism or component thereof.

“Cell quality indicator” refers to an indicator of cellular blood component quality. Exemplary cell quality indicators are parameters corresponding to the physical state of a fluid containing cells or cellular blood components that provide a measurement useful for assessing its quality for subsequent use in therapeutic applications. During metabolism, cells consume glucose and generate two lactate molecules for each glucose molecule consumed. The lactate formed has the effect of lowering the pH of the blood component sample. As a finite amount of glucose is provided to cells during storage, stored cellular blood components which consume glucose too quickly are degraded. Lower glucose consumption rates and lactate production rates are indicative of cellular blood components that retain a high therapeutic effectiveness when stored. Therefore, low glucose consumption rates and lactate production rates are considered indicator of high cell quality.

“Flux of photons” or “photon flux” refers to the number of photons of light passing a defining area at a given time. Typically, photon flux is defined in units of: (number of photons) cm⁻² s⁻¹.

“Polymer” refers to a molecule comprising a plurality of repeating chemical groups, typically referred to as monomers. Polymers are often characterized by high molecular masses. Polymers useable in the present invention may be organic polymers or inorganic polymers and may be in amorphous, semi-amorphous, crystalline or partially crystalline states. Polymers may comprise monomers having the same chemical composition or may comprise a plurality of monomers having different chemical compositions, such as a copolymer. Cross linked polymers having linked monomer chains are particularly useful for some applications of the present invention. Polymers useable in the methods, devices and device components of the present invention include, but are not limited to, plastics, elastomers, thermoplastic elastomers, elastoplastics, thermostats, thermoplastics. Exemplary polymers include, but are not limited to, poly(vinyl chloride).

In the following description, numerous specific details of the devices, device components and methods of the present invention are set forth in order to provide a thorough explanation of the precise nature of the invention. It will be apparent to those of skill in the art, however, that the invention can be practiced without these specific details.

This invention provides methods, devices and device components for treating biological samples with electromagnetic radiation. The methods, devices and device components of the present invention are capable of providing well characterized, uniform and reproducible net radiant energies and/or radiant powers to biological samples undergoing processing. In addition, the present methods, devices and device components are capable of delivering electromagnetic radiation to biological samples having a distribution of wavelengths selected to provide enhanced pathogen reduction, while minimizing photoinduced damage to components comprising therapeutic and/or reinfusion agents.

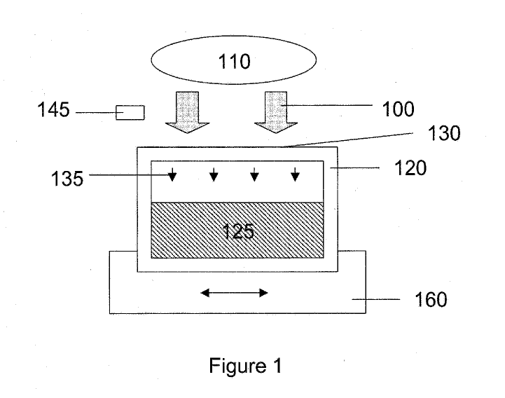

FIG. 1 shows a schematic diagram illustrating a method and apparatus for reducing pathogens in blood or blood component held in a container comprising poly(vinyl chloride) and a citrate plasticizer (i.e. a citrate plasticized PVC container). As shown in FIG. 1, electromagnetic radiation (schematically illustrated by arrows 100) is generated by source of electromagnetic radiation 110 and is directed onto a container 120 comprising poly(vinyl chloride) and a citrate plasticizer. Container 120 holds a blood or blood component sample 125 undergoing pathogen reduction treatment which may optionally comprise one or more added anticoagulant agent, enhancer, photosensitizer, preservative or diluent. Container 120 also has at least one partially transparent surface 130 which at least partially transmits electromagnetic radiation (schematically illustrated as arrows 135) having a selected distribution of wavelengths, for example electromagnetic radiation capable of directly reducing pathogens and/or inducing chemical reactions resulting in pathogen reduction. Electromagnetic radiation 135 having the selected distribution of wavelengths is transmitted through container 120 and is at least partially absorbed by blood or blood component sample 125, thereby reducing the biological activity of pathogens present. Optionally, agitator 160 is provided for mixing blood or blood component sample 125 during exposure to electromagnetic radiation to ensure that the electromagnetic radiation is uniformly provided to all components of the sample undergoing treatment. Agitator 160 may be operably connected to container 120 using any means known in the art of fluid processing.

Optionally, the transmission characteristics (percentages transmission and/or extinction coefficients) of partially transparent surface 130 of poly(vinyl chloride) and citrate plasticizer container 120 are well characterized (e.g. measured and/or calculated) prior to treatment of blood or blood component sample 125. In one embodiment of this aspect of the invention, the radiant power of electromagnetic radiation 100 generated by source of electromagnetic radiation 110 is continuously, periodically or intermittently monitored by photodetector 145 positioned in optical communication with source of electromagnetic radiation 110. This arrangement allows the radiant powers and/or net radiant energies actually delivered to blood or blood component sample 125 to be accurately calculated with knowledge of the surface area and transmission characteristics of partially transparent surface 130 of poly(vinyl chloride) and citrate plasticizer container 120.

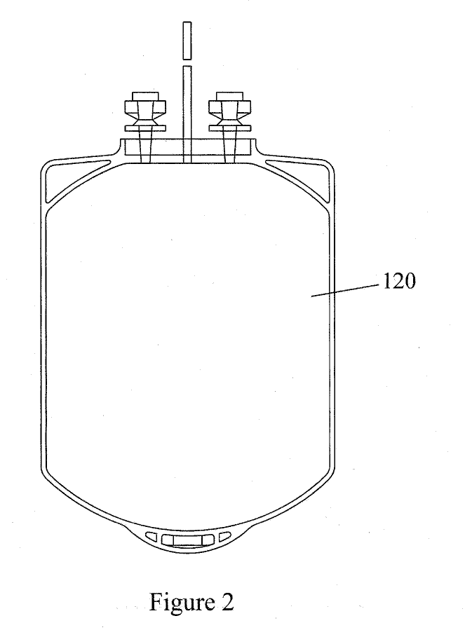

FIG. 2 provides a schematic diagram of an exemplary container 120 comprising an at least partially transparent citrate plasticized poly(vinyl chloride) bag for holding a blood or blood component sample. Citrate plasticized poly(vinyl chloride) bag comprises a citrate plasticized poly(vinyl chloride) film (Specific Gravity: 1.19±0.02) made of n-butyryltri-n-hexyl citrate (C₂₈H₅₀O₈; Molecular weight equal to 514 atomic mass units) with a percentage by weight equal to about 38%. The citrate plasticized poly(vinyl chloride) bag has a volume of 1 liter, a width equal to 6.75±0.25 inches and length equal to about 9.50±0.25 inches. The walls of the citrate plasticized poly(vinyl chloride) bag have a thickness equal to 0.015±0.001 inch. In some treatment processes citrate plasticized poly(vinyl chloride) bag holds a blood or blood component sample having a volume selected from the range of about 200 milliliters to about 400 milliliters and a surface area of the citrate plasticized poly(vinyl chloride) bag equal to about 347 cm² per side is illuminated during treatment.

The composition and physical dimensions of citrate plasticized poly(vinyl chloride) bag provide a number of beneficial attributes for processing blood. The citrate plasticized poly(vinyl chloride) bag is photolytically stable and does not undergo significant changes during a treatment protocol in the percentages of transmission (or extinction coefficients) corresponding to light of the effective wavelength for a given process. The citrate plasticized poly(vinyl chloride) bag also significantly transmits (i.e. has percentage transmission greater than about 30%) light having wavelengths ranging from 285 nanometers to 365 nanometers, which corresponds to a wavelength range useful for processing platelet-containing samples. The citrate plasticized poly(vinyl chloride) bag has tensile strengths of 2000 PSI (machine direction; minimum) and 1900 PSI (transverse direction; minimum) and is capable of elongation (290% (machine direction; minimum), 330% (transverse direction; minimum).

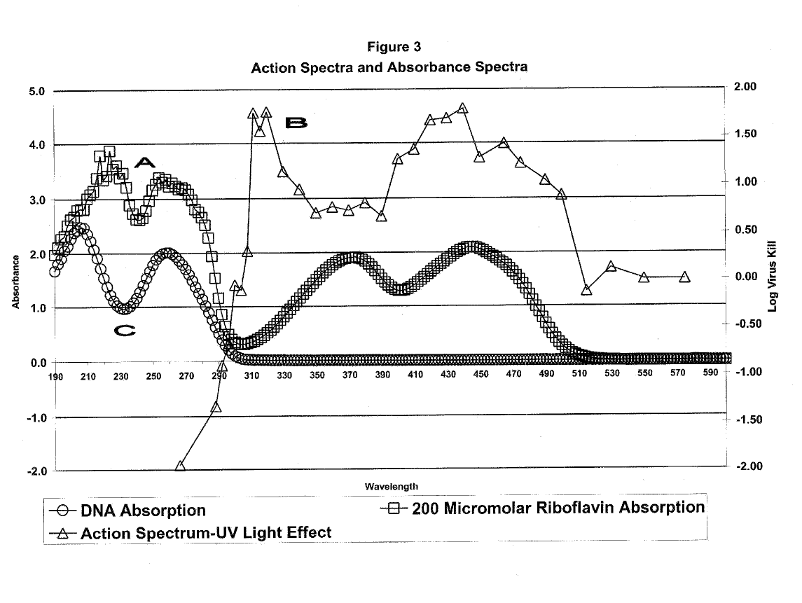

In an embodiment useful for reducing pathogens in blood or blood components having an added 7,8-dimethyl-10-ribityl isoalloxazine photosensitizer, the selected distribution of wavelengths includes wavelengths of electromagnetic radiation absorbed by 7,8-dimethyl-10-ribityl isoalloxazine in bound or unbound states in the biological sample. Absorption of electromagnetic radiation by the 7,8-dimethyl-10-ribityl isoalloxazine present in a blood or blood component sample initiates photochemical reactions resulting in a reduction of the biological activities of pathogens. FIG. 3 shows an absorption spectrum of a 200 micromolar solution of 7,8-dimethyl-10-ribityl isoalloxazine in phosphate buffer saline (absorbance vs. wavelength; curve A) which is characterized by absorption maxima at about 370 nanometers and about 450 nanometers. The absorption spectrum of 7,8-dimethyl-10-ribityl isoalloxazine, however, is expected to change when it is bound to biological molecules, such as proteins, RNA molecules or DNA molecules, present in a biological sample. FIG. 3 also shows an action spectrum (log virus kill; curve B) corresponding to the reduction efficiency of a platelet and plasma-containing sample having 7,8-dimethyl-10-ribityl isoalloxazine and exposed to selected wavelengths of ultraviolet and visible electromagnetic radiation. FIG. 3 also shows a DNA absorption spectrum (absorbance vs. wavelength; curve C). From the action spectrum provided in FIG. 3, it is likely that 7,8-dimethyl-10-ribityl isoalloxazine present in plasma-containing samples and plasma containing samples has its absorbance maxima shifted to higher wavelengths (about 430 nanometers and about 470 nanometers). Accordingly, exemplary pathogen reduction methods for platelet and/or plasma-containing blood components use electromagnetic radiation having a distribution of wavelengths has wavelengths ranging from about 300 nanometers to about 500 nanometers. The present invention also includes pathogen reduction methods wherein the distribution of wavelengths corresponds to electromagnetic radiation which is capable of directly reducing the biological activities of pathogens present in the sample (i.e. when no photosensitizer is present in the biological sample).

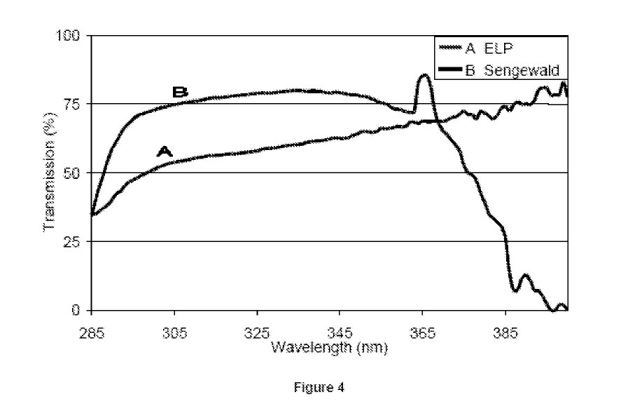

FIG. 4 shows transmission spectra of a citrate plasticized poly(vinyl chloride) bag having n-butyryltri-n-hexyl citrate (38% weight percent) (curve A) and a conventional polyolefin bag (curve B). As shown in FIG. 4, use of the citrate plasticized poly(vinyl chloride) bag reduces transmission of light in the short wavelength region (285-305 nm) relative to the polyolefin bag. This difference in transmission spectra is advantageous for blood processing applications for blood components comprising therapeutic agents or reinfusion agents because light in this short wavelength region is known to damage to cellular components, such as platelets and cellular proteins, and noncellular blood components, such as plasma proteins. Indeed, the reduction effect of short wavelength UV light is important in order to avoid severe damage to treated platelet organelles such as mitochondria which maintains part of bioenergy ATP supply for platelet viability and function. Referring again to FIG. 4, use of the citrate plasticized poly(vinyl chloride) bag also increases transmission of light at relatively long wavelengths (365-400 nm) relative to the polyolefin bag. This difference in transmission spectra is advantageous for blood processing applications using a 7,8-dimethyl-10-ribityl isoalloxazine photosensitizer, because this compound has an absorbance maximum in this region of the electromagnetic spectrum when in free or bound states.

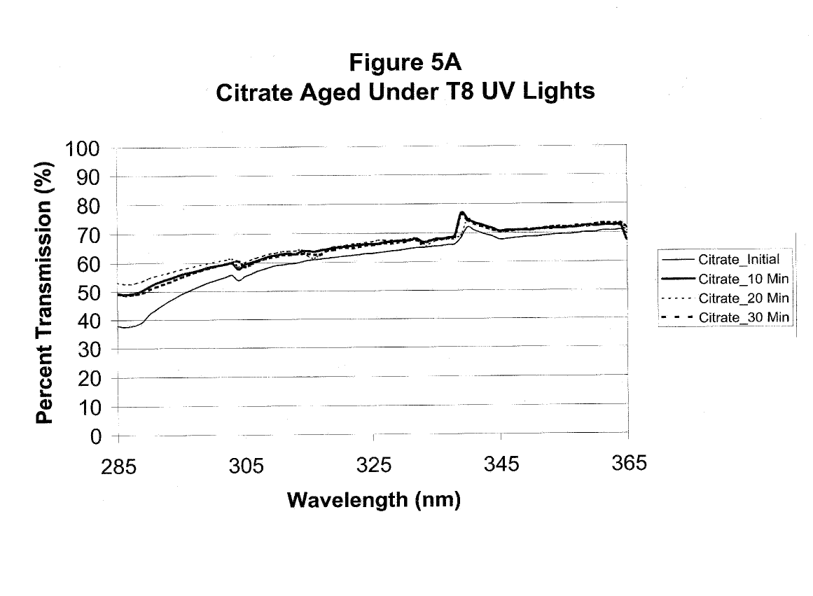

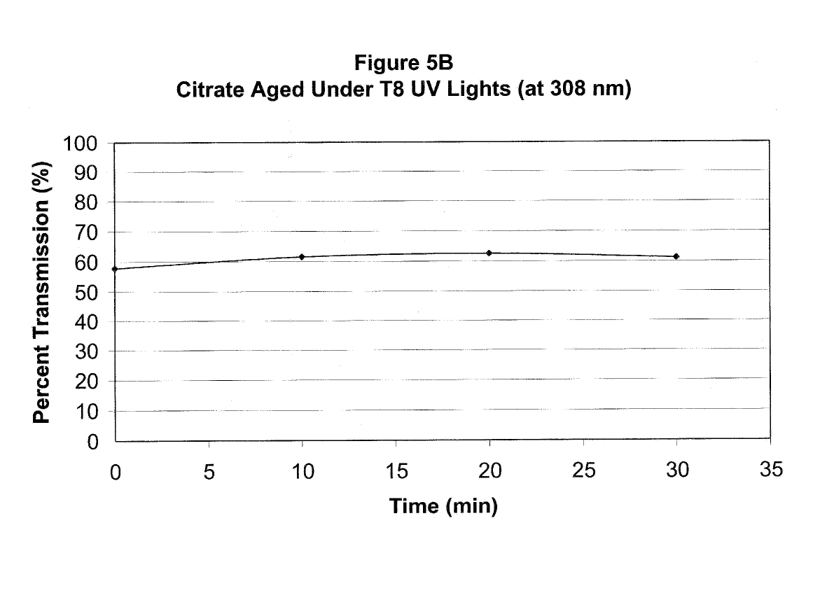

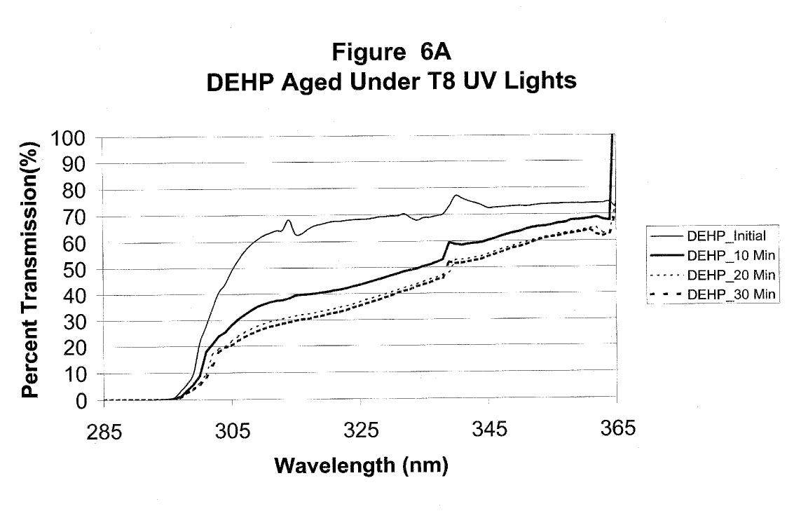

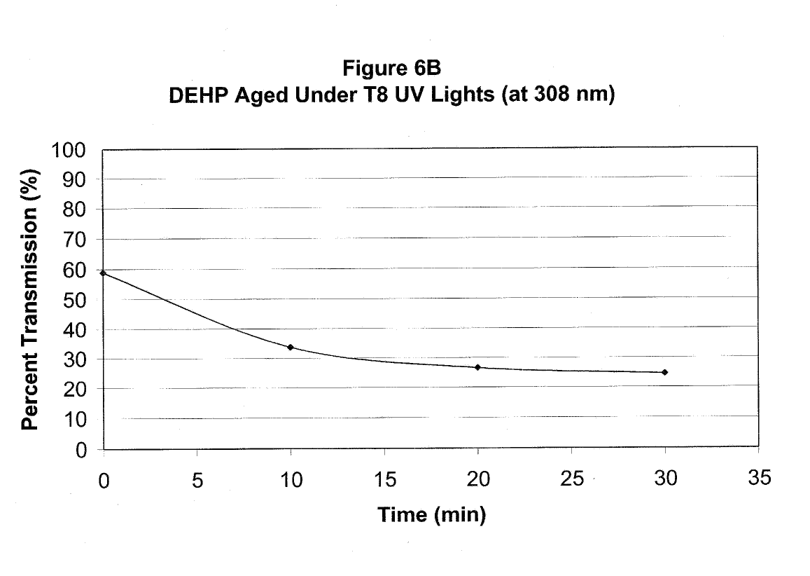

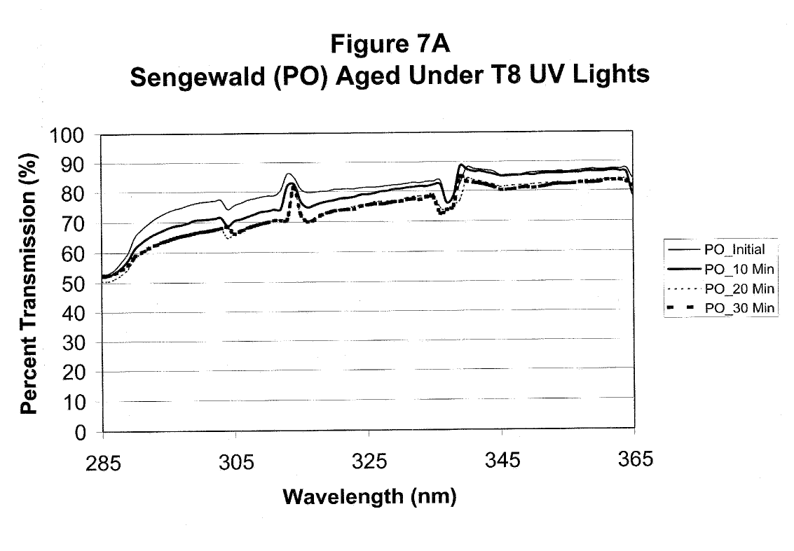

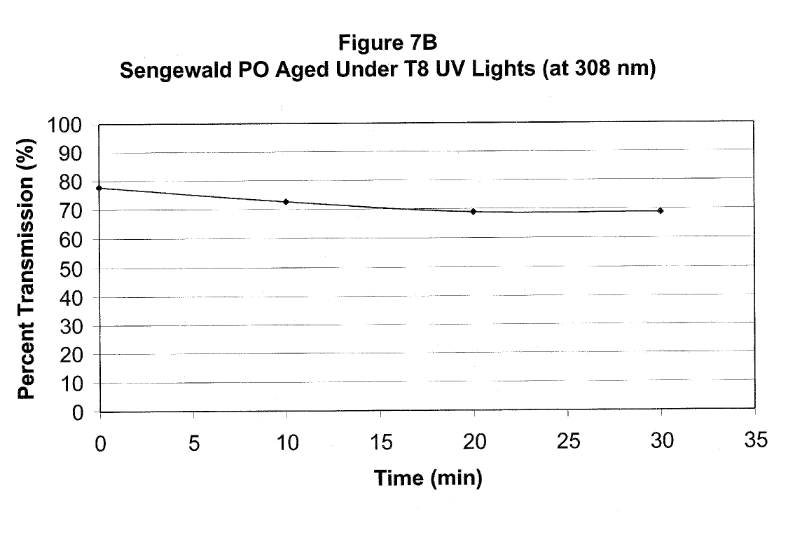

FIG. 5A shows transmission spectra of a citrate plasticized poly(vinyl chloride) bag upon exposure to ultraviolet radiation for several illumination times and FIG. 5B provides a plot of the percentage transmission at 308 nanometers as a function of exposure time. FIG. 6A shows transmission spectra of a poly(vinyl chloride) and DEHP plasticizer bag upon exposure to ultraviolet radiation for several illumination times and FIG. 6B provides a plot of the percentage transmission of this bag at 308 nanometers as a function of exposure time. FIG. 7A shows transmission spectra of a polyolefin bag upon exposure to ultraviolet radiation for several illumination times and FIG. 7B provides a plot of the percentage transmission of this bag at 308 nanometers as a function of exposure time. The data in FIGS. 5A, 5B, 6A, 6B, 7A and 7B were generated by exposing bags having different compositions to a source of electromagnetic radiation providing a substantially constant radiant output with an intensity of about 10.5 mW/cm² as measured by a 320 nm OAI powermeter (Optical Associates Inc., San Jose, Calif.). The source of electromagnetic radiation was an Ushio G25T8E Nichia NP-803 phosphor (radiant wavelengths=265 nm to 375 nm; peak wavelength=306 nm-308 nm). Exposure times were 0 minutes, 10 minutes, 20 minutes and 30 minutes. The bags investigated were moved out of optical communication with the source of electromagnetic radiation after the indicated exposure times. The bags investigated were then placed on an integrating sphere and exposed to a constant radiant source with an intensity of about 5.4 mW/cm² as measured by a 320 nm OAI powermeter. The spectral output/transmission characteristics were measured by the OL-754 Spectroradiometer (Optronic Laboratories, Inc., San Diego, Calif.).

As shown in FIGS. 5A and 5B, the citrate plasticized poly(vinyl chloride) bag exhibits a less than about 10% increase in percentage transmission at 308 nanometers during illumination for an exposure time of 30 minutes. In contrast, the transmission spectra of the poly(vinyl chloride) and DEHP plasticizer container, as shown in FIGS. 6A and 6B, exhibits a more than about 55% decrease in percentage transmission at 308 nanometers for an exposure time of 30 minutes. As shown in FIGS. 7A and 7B, the polyolefin bag exhibits a more than about 10% decrease in percentage transmission at 308 nanometers for an exposure time of 30 minutes. A comparison of the transmission spectra provided in FIGS. 5A, 5B, 6A, 6B, 7A and 7B shows citrate plasticized poly(vinyl chloride) bags are particularly photolytically stable and do not to undergo significant photoinduced decomposition or degradation during treatment of a sample with electromagnetic radiation. Therefore, it is expect that use of a poly(vinyl chloride) and citrate plasticizer containers in the methods of the present invention provides significantly more uniform and reproducible radiant energies and/or radiant powers to biological sample than conventional container for biological samples, such as poly(vinyl chloride) with a DEHP plasticizer bags and polyolefin bags.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. Methods and devices useful for the present methods can include a large number of optional device elements and components including, optical filters such as bandpass filters, high pass cutoff filters and low pass cutoff filters, collimation elements such as collimating lenses and reflectors, focusing elements such as lens and reflectors, reflectors, diffraction gratings, flow systems, fluid mixing systems such as stirrers and shakers, fiber optic couplers and transmitters, temperature controllers, temperature sensors, broad band optical sources, narrow band optical sources, fluid control elements such as peristaltic pumps, valves, filters, centrifuge systems, elutriation systems and combinations of these elements.

All references cited in this application are hereby incorporated in their entireties by reference herein to the extent that they are not inconsistent with the disclosure in this application. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques specifically described herein are intended to be encompassed by this invention.

### EXAMPLE 1

### Platelet Viability Studies Using the Present Pathogen Reduction Methods

Background:

Changes in several in vitro platelet quality parameters during platelet storage have been associated with decreased in vivo platelet viability measured by radiolabeled platelet recovery and survival post-transfusion. The purpose of this study focused on identifying the correlation of in vitro parameters with platelet in vivo recovery. We then verified the predictability of the in vitro cell quality measures for in vivo recovery of platelets treated with a pathogen reduction process using riboflavin and light.

Study Design and Methods:

Two platelet recovery clinical studies using radiolabelled platelets were performed under regulatory review and approval. In the first study, a correlation of in vitro cell quality parameters was established with in vivo platelet recovery using 18 platelet products collected by a Trima apheresis procedure, treated with various doses of UV light and stored for 5 days. Using predictors of in vivo recovery based on lactate production and pH, a novel process designed for pathogen reduction of platelet products using riboflavin and light (Mirasol PRT) (6.2 J/mL+50 μM riboflavin) was developed. The predictability of lactate production and pH for in vivo recovery was then verified through direct testing with PRT treated platelets in a subsequent human clinical trial.

Results:

UV treatment increased lactate production, glucose consumption and P-selectin expression, and resulted in decreased pH, HSR and swirl during storage. This behavior was exhibited in a UV-dose dependent manner. All of the changes in cell quality parameters were correlated with platelet in vivo recovery. Among them, lactate production and pH were identified by linear regression analysis as parameters most strongly correlated to platelet in vivo recovery. The correlation coefficients for lactate production and pH were 0.9090 and 0.8831 with p values of 0.007 and 0.031, respectively. Similar correlations of lactate production and pH with platelet survival and the same trend of prediction were also observed. The day-5 platelet recovery value predicted from these algorithms was 44-55% for platelets treated with Mirasol PRT. A subsequent clinical study with 24 platelet products demonstrated that the in vivo recovery of PRT treated platelets was 51.4±18.6 percent, a value well within the range of this prediction.

Conclusion:

These results demonstrate that platelet in vivo recovery can be predicted from in vitro cell quality parameters and that under the conditions utilized here, lactate production and pH are the most relevant in vitro indicators for PRT treated platelet viability in vivo.

Platelet transfusion therapy still remains a mainstream in preventing or treating bleeding episodes for thrombocytopenic patients or patients with high-risk of bleeding. Success in platelet transfusion depends on the cellular viability and hemostatic activity of the transfused product and on the physiological status of the transfusion recipient. While the physiological status of the recipient is reflected by the ability of the recipient to tolerate the transfused platelets and the propensity to clear them from the circulation through the reticuloendothelial system, cell viability is often determined in autologous donors by in vivo recovery and survival post-transfusion of radiolabeled platelets. Though better platelet recovery is normally associated with a longer platelet survival time, in vivo recovery is more often used in measuring platelet transfusion efficacy. For the past few decades platelet viability during storage has improved significantly by optimizing the storage conditions such as temperature, gas exchange of the storage container and agitation. However platelet products stored under current blood banking conditions still demonstrate a storage time-dependent reduction in their in vivo viability, primarily due to the development of a platelet storage lesion. Thus determination of in vivo cell viability becomes a critical step in developing any new technology for platelet production, processing and storage and in quality control of currently used platelet products.

Evaluation of cell viability in vivo using a method of radiolabeling test platelets has proven to be a challenging task as in vivo human clinical trials are expensive, time-consuming and expose donors to radioactivity. Given the fact that the reduction in in vivo cell viability is always associated with significant changes in in vitro cell quality tests, the possibility of using in vitro tests to predict in vivo viability has been extensively explored. In early studies on platelet storage at low temperature, in first-generation containers, and by freezing, it was observed that a platelet morphology change from discoid shape to spherical form during platelet storage had been accompanied with low platelet recovery. The observation provided a basis for using platelet swirl to predict platelet viability. Though scoring swirl is one of the simplest lab methods available, it is a qualitative test and lacks sensitivity and reproducibility from lab to lab. Assays on extent of shape change and response to hypotonic shock are quantitative and showed much better correlations with the in vivo recovery with correlation coefficients (r) of 0.71 and 0.57, respectively. Metabolic parameters for platelets such as lactate production and pH change were also shown to have a significant correlation with platelet recovery and survival. Measurement of the correlation of P-selectin expression, a platelet activation marker, with in vivo recovery has yielded inconsistent results. Holme et al reported a poor correlation with platelet recovery¹¹ while others found significant correlations. The clinical utility of P-selectin expression as a reliable predictor has been questioned by the findings that neither mouse platelets genetically lacking P-selectin nor human thrombin-activated platelets fully expressing P-selectin had different in vivo lifespans from normal and resting platelets. Platelet apoptosis has also been shown to attribute to the development of the platelet storage lesion, but direct relevance to in vivo cell viability has not been established.

All studies mentioned above have analyzed the correlations of various in vitro platelet quality parameters with in vivo platelet viability and have concluded that some of the parameters may be possible predictors of in vivo recovery. None of these studies have performed a direct verification of their findings. The purpose of this study focused on identifying in vitro cell quality parameters with the best correlations with platelet recovery for platelets treated with UV light at various doses. Platelets were treated in a polyolefin bag and then stored for 5 days in a citrated PVC bag. Using the identified cell quality parameters and their correlation with in vivo recovery, we predicted a range of in vivo recovery for the platelets treated with a novel pathogen reduction process known as Mirasol PRT. The prediction was subsequently verified in a clinical trial conducted in the United States under the auspices of an IDE. From this work, we identified and further verified that lactate production and pH were the best predictors for platelet recovery. These observations are further indications that in vitro measures of cell quality can be predictive of in vivo outcomes and afford valuable approaches for the pre-clinical evaluation of new platelet processing methodologies.

Materials and Methods

Trima-Collected Apheresis Platelet Concentrate Preparation

All platelet products in the studies were apheresis platelet concentrates from a single donor collected in 1-liter citrate plasticized poly(vinyl chloride) bag having n-butyryltri-n-hexyl citrate −38% weight percent (“citrated PVC ELPTm bag”) by the local blood centers using a TRIMA Automated Blood Component Collection System (Gambro BCT, Lakewood, Colo.). In clinical study one a target platelet yield was 3.51×10¹¹. Whereas the target yield was 4.42×10¹¹ platelets in the 2^{nd} clinical study.

Clinical Study One:

The study was conducted at the Department of Haematology & Cell Biology, Faculty of Health Sciences, University of the Orange Free State (Bloemfontein, South Africa) under reviews and approvals of the Ethics Committee of the University of the Orange Free State and the South African Medicine Control Council (MCC). Upon completing informed consent forms, study volunteers at age from 18 to 65 years old were screened and selectively enrolled in the study based on the local criteria and AABB requirements for platelet donation.

UV Light Treatment and Platelet Storage

The platelet concentrate with a volume of 250 mL was transferred into a 3-litre polyolefin bag (Sengewald, Rohrdorf, Germany), followed by addition of 27 mL sterile 500 μM riboflavin so that the final concentration in the product was ca. 50 μM. The platelet products were then exposed to UV light (phosphor 265-370 nm) at either a medium dose level (7.2 J/ml) or high dose level (12.4 J/ml). Total illumination time varied from approximately 5-10 minutes with agitation at a temperature of 25-30° C. After treatment, platelet products were transferred into a citrated polyvinyl chloride ELP™ bag (Gambro BCT, Lakewood, Colo.). The treated and control PCs were stored for an additional 5 days at 20-24° C. under standard blood bank conditions. Control platelet products were prepared in the same manner as the treated counterparts except no riboflavin was added and no UV light treatment was performed.

In Vitro Cell Quality Tests

Platelet samples were taken for lab tests at day 0, 3 and 5 of platelet storage using aseptic technique and analysis was completed within 2 hours. The in vitro cell quality tests for platelet count, swirl score, pH, pO₂, pCO₂, lactate and glucose were performed per standard operating procedures (SOPs) of the trial site. Hypotonic shock response (HSR) and P-selectin expression were measured as described by Ruane et al. (Ruane P H, Edrich R, Gampp D et al. Photochemical inactivation of selected viruses and bacteria in platelet concentrates using riboflavin and light. Transfusion 2004;44:877-85.)

In Vivo Platelet Recovery and Survival Measurement

At the end of the 5-day storage period, a small aliquot of the platelets was radiolabeled with ¹¹¹Indium, according to the study site's SOP (in agreement with local and international standards for radiolabeling of human platelets). The labeling procedure was performed after formation of ¹¹¹In-tropolonate through mixing of ¹¹¹Indium chloride (Amersham) with tropolone. If the pH of the platelet samples to be labeled was>6.5, it was brought down to 6.5 to prevent irreversible aggregation of platelets during pelleting.

After washing and resuspension in plasma, a radiolabeled aliquot was infused into the autologous donor. The total radioactivity that was given to a subject in this study was less than 8 MBq. Blood samples for radioactivity counting were collected 15 minutes, 1 hour and 2-3 hours after infusion, twice (AM and PM) on day 1 post-infusion and once on days 2-6 post-infusion. After correcting for 2-hour radioelution as described by Holme, et al., (Holme S, Heaton A, Roodt J. Concurrent label method with 111In and 51Cr allows accurate evaluation of platelet viability of stored platelet concentrates. Br J Haematol 1993;84:717-23.) in vivo radiolabeled platelet recovery and survival values were calculated by the COST computer program using the multiple-hit model.

Clinical Study Two:

The second clinical study focusing on verifying in vivo recovery of platelets treated with the Mirasol PRT process was performed at Dartmouth-Hitchcock Medical Center of New Hampshire and Norfolk Red Cross Center of Virginia. The study was reviewed and approved by the Institutional Review Board (IRB) for both clinical study sites and the United States FDA under an Investigational Device Exemption (IDE). Upon completion of the informed consent form, each participating volunteer was screened for eligibility based on all FDA and AABB criteria for platelet donation and then were selectively enrolled into the study.

Mirasol PRT Treatment and Platelet Storage

Within 2-8 hours after platelet apheresis collection, a volume of 250 mLs of platelets was gravimetrically transferred from the collection ELP bag to a separate illumination and storage ELP container. Riboflavin solution (500 μM) at a volume of 28 mL was added to the test product through a sterile barrier filter with a syringe. The product was placed in the Mirasol PRT Illumination device manufactured by Navigant Biotechnologies, Inc. (Lakewood, Colo.) and exposed to 6.2 J/mL dose of ultraviolet light. The control products had no riboflavin added and were not treated with UV light. After the MIRASOL PRT process, the products (control and test) were stored under normal blood banking conditions of 22±2° C. with horizontal agitation for 5 days.

Radiolabeling and In Vivo Platelet Recovery and Survival Measurement

At the end of the 5-day storage period, an aliquot of platelet product was radiolabeled with ¹¹¹In-oxine, using the procedure specified by Holme, et al. A 2-hour radioelution evaluation on each radiolabeled sample was performed as described by Holme, et al. (Holme S, Heaton A, Roodt J. Concurrent label method with 111In and 51Cr allows accurate evaluation of platelet viability of stored platelet concentrates. Br J Haematol 1993;84:717-23.)

An aliquot (approximately 2-10 mL) of ¹¹¹In-radiolabeled platelets (control or test) were re-infused into the original subject. Blood samples (5 mL into EDTA tubes) were drawn for measurement of radioactivity at 1, 3, 15 and 26 hours, 2, 3, 4, 5-6, 7 and 10 days post-infusion. In vivo radiolabeled platelet recovery and survival values were calculated by COST computer program using the multiple-hit model after correction for radioelution.

UV Light Transmission Test

Statistics

For all in vitro cell quality parameters, means and standard deviations were calculated. Statistical comparisons were performed using analysis of covariance (ANCOVA) for repeated measurements where applicable. This analysis was performed using ‘proc mixed’ in SAS v 8.1. Sequence effects were initially included in the model but dropped if non-significant.

Results

Clinical Trial One: UV Treatment Accelerates Cellular Glycolytic Metabolism

Following Ethical Committee approval and notification of the MCC, the competent authority of South Africa, a total of 18 platelet products were collected with yields ranging from 2.9 to 3.8×10¹¹ platelets using standard Trima apheresis procedures. Products were treated in a polyolefin container, Sengewald bag, with UV light at either a medium UV dose (7.2 J/ml; N=5) or a high UV dose (12.4 J/ml; N=6) in the presence of 50 μM riboflavin on the day of collection. An additional seven products served as controls which were not treated with UV light. All treated and control PC products were transferred to and stored in ELP™ bags under normal blood bank conditions for 5 days post-apheresis collection. At day 0 (pre-treatment), day 3 and day 5 of storage, the samples from all PC products were measured for pH, pO₂, pCO₂, lactate and glucose concentrations, P-selectin, HSR and platelet swirl. Table 1 summarizes the results of these cell metabolic and quality measurements. Treated platelets, when compared to control platelets, showed increases in lactate production and glucose consumption accompanied with a decrease in sample pH during storage, indicating that UV light treatment increased cellular glycolytic metabolism. UV treatment also accelerated an increase in P-selectin expression and decreases in HSR and swirl score during platelet storage. It appears that the severity of these changes during platelet storage were in direct proportion to the levels of UV dose applied.

Correlation of In Vitro Platelet Quality With In Vivo Cell Recovery and Survival

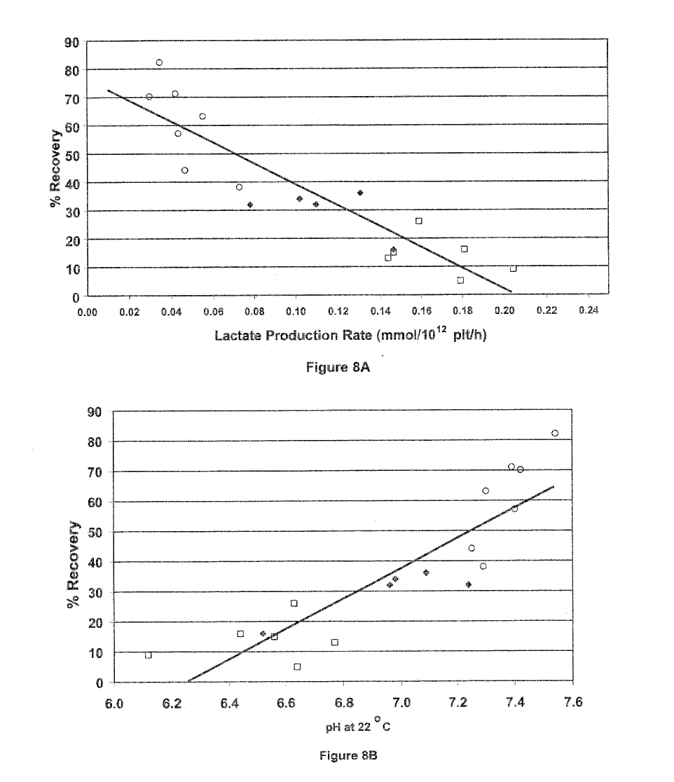

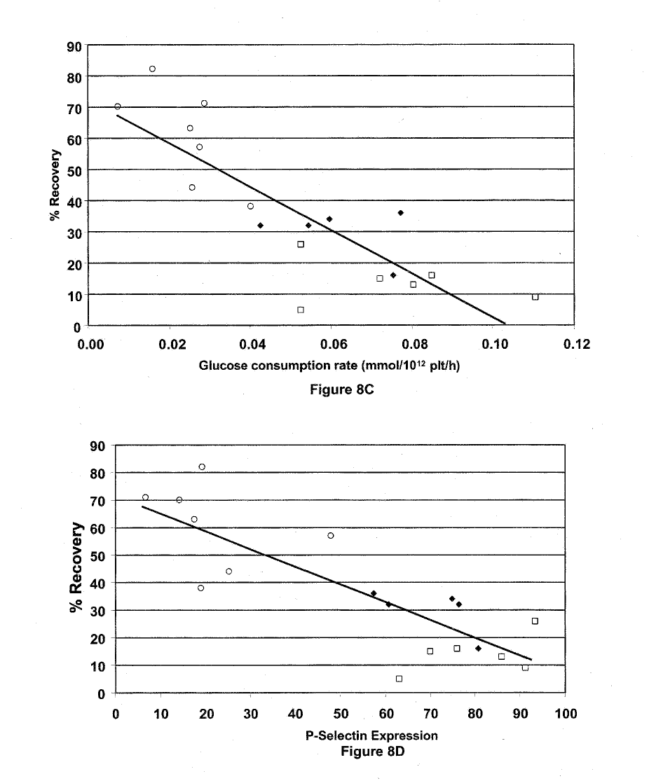

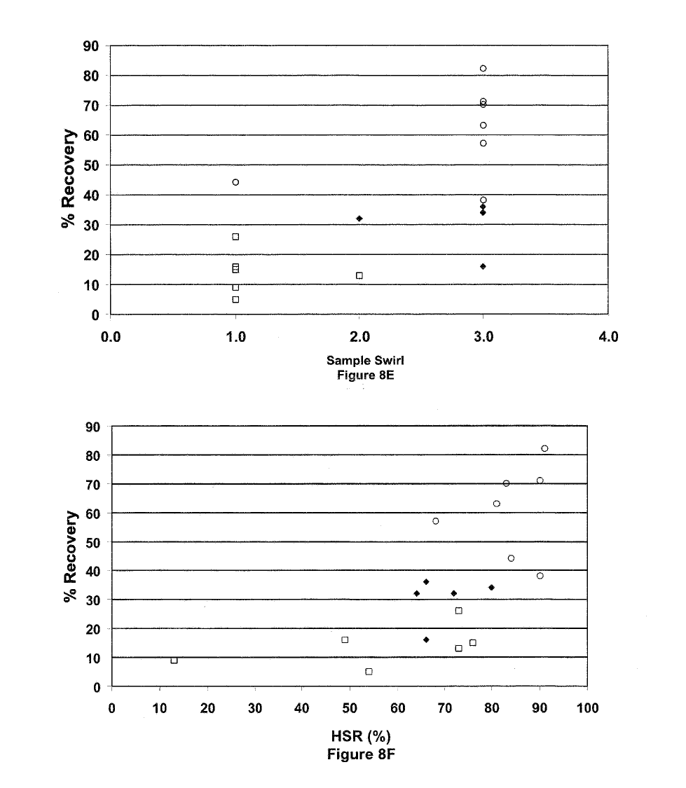

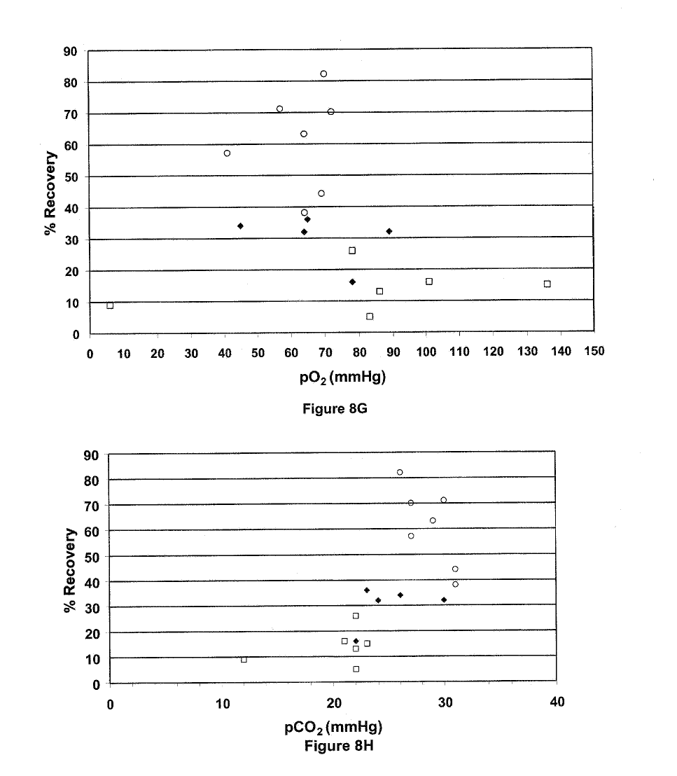

At the end of 5-day storage, all treated and control platelet samples were radiolabeled with Indium followed by infusion of the labeled platelets into the same donor. The radioactivities of infused platelets in vivo were measured for up to seven days and in vivo recovery of infused platelets was calculated using a multiple-hit model analysis. The averages in vivo recoveries were 60% (SD=16), 30% (SD=8) and 14% (SD=7) for the platelets treated with zero, medium and high doses of UV light, respectively. FIGS. 8A-H show correlations of in vitro cell quality parameters with in vivo platelet recovery. The in vivo platelet recovery is function of values of lactate production (a), pH at 22° C. on day 5 (b), glucose consumption (c), P-selectin expression percent on day 5 (d), swirl score on day 5 (e), HSR percent on day 5 (f), pO₂ (g) and pCO₂ on day 5 (h). In the graph the open circle is control platelets, solid diamond medium dose of UV light treated platelets and solid square high dose of UV light treated platelets. In FIGS. 8A-H, each metabolic and cell quality parameter measured during storage or at day 5 is plotted against the platelet recovery for every individual platelet product. A clear correlation of the parameters with the in vivo recovery was observed. Among the parameters plotted in the graphs, lactate production, pH, glucose consumption and P-selectin appeared to have good correlation with the measured in vivo recovery. The degree of these correlations was quantified by linear regression analysis and is summarized in Table 2. Both correlation coefficient r-values and F-values identified lactate production rate and pH to be most significantly correlated with in vivo platelet recovery with p values of 0.007 and 0.031, respectively. The remaining parameters, in order of their extent of correlation to in vivo recovery, were glucose consumption rate, P-selectin expression, HSR, Swirl, pCO₂ and pO₂.

A very similar pattern for correlation of each cell quality parameter with platelet survival time was observed using the same liner regression analysis approach. Again, lactate production and pH were identified to possess the strongest correlation with platelet survival. However F-values for these determinations indicated significantly higher levels of variation than those observed for recovery values. For this reason, algorithms used for platelet survival were deemed to be less reliable predictors of platelet survival.

Verification of Lactate and pH as Predictors of In Vivo Recovery: Clinical Trial Two

Information obtained from the first clinical study conducted in South Africa was used to design the platelet processing conditions for the Mirasol PRT treatment procedure. Under these conditions, Mirasol PRT treatment maximized pathogen reduction capabilities without compromising platelet therapeutic values as evaluated by a series of in vitro cell quality tests during platelet storage. Unlike the platelet processing scheme described in the first clinical study, fresh platelets in this study were processed with Mirasol PRT by exposing them to 6.2 J/mL UV light in the presence of 50 μM riboflavin in a citrated polyvinyl chloride ELP™ bag. Products were then stored in the same bag for 5 days post-treatment, eliminating the need for a bag transfer step. The use of the ELP bag had an advantage over the Sengewald bag used in the first clinical study via a significant reduction in light transmission in the short wavelength region (285-305 nm) and increased transmission at relatively long wavelengths (365-400 nm), as illustrated in FIG. 4. The region with long wavelengths corresponds to the area in which riboflavin has maximal absorption. The effect of the Mirasol PRT treatment conditions used in this study on in vitro platelet cell quality and on viral and bacterial inactivation has been extensively evaluated, as reported by Li et al (Li J, Xia Y, Bertino AM et al. The mechanism of apoptosis in human platelets during storage. Transfusion 2000;40:1320-9.) and Ruane et al.

Using the linear regression equations for lactate rate and pH obtained from the first clinical study (Table 2), the platelet recovery for samples treated with the standard operating conditions of the Mirasol PRT process was predicted to be between 44-55% (Table 3). The values for lactate production rate and pH that were used in generating this prediction were derived from previous in vitro studies of platelet performance following treatment of products at 6.2 J/mL in an ELP container and subsequent storage for 5 days. The reliability of using lactate rate and pH parameters for predicting in vivo recovery was verified in the second trial of the Mirasol PRT process. A total of 24 platelet products collected on the Trima platform were used. After UV treatment with 6.2 J/mL and 5-day storage in an ELP bag, the treated platelets demonstrated an average recovery of 51.4% with standard deviation 18.6%. The observed in vivo recovery values for treated products ranged from 24.3% to 95.8%. This result demonstrated that lactate production rate and pH parameters provided a simple and reliable means for predicting platelet recovery in vivo.

Discussion

Many attempts have been made to predict platelet in vivo viability from in vitro cell quality parameter measurements. Success in using these predictions has been only sparsely reported, probably due to the limitation of relatively poor correlations between these parameters and in vivo measurements of recovery. The challenge stems both from a considerably large variation in in vivo recovery measurement within normal volunteers, which is mainly due to recipient physiological status, and from biological variability in the in vitro tests for platelet products. ²⁴The extent of correlations also depends on the range and distribution of each variable. The wider the range and more even the distribution of values for a cell quality parameter that are obtained, the better the correlations which can be observed. To broaden the range of cell quality values that could be observed in this study, platelets were treated with three different doses of UV light and stored for 5 days in the first clinical study. Results showed that all measured in vitro cell quality parameters responded to UV light treatment in a dose dependent manner. UV treatment increased lactate production, glucose consumption and P-selectin expression, and resulted in decreased pH, HSR and swirl during storage. All of these changes in cell quality parameters were correlated with platelet in vivo recovery to varying degrees. Among them, lactate production and pH were identified by linear regression analysis as parameters most strongly correlated to platelet in vivo recovery. The correlation coefficients for lactate production and pH were 0.909 and 0.883 with p values of 0.007 and 0.031, respectively. A similar pattern for correlation of in vitro cell quality with platelet survival was also observed. The value of these prediction algorithms was successfully verified through a subsequent clinical study which demonstrated that the observed platelet in vivo recovery was well within the range of predicted values. These results demonstrate that platelet in vivo recovery is predictable from in vitro cell quality parameters as has been suggested previously suggested and that under the conditions utilized here, lactate production and pH are the most relevant in vitro indicators for PRT treated platelet viability in vivo.

Our observations reported here are consistent with previous work. Lactate is a final metabolic product in the platelet glycolytic pathway, and is converted to lactic acid and released into the storage medium during storage. Lactate accumulation directly reflects the status of platelet glycolytic flux while UV treatment stimulates the lactate production rate in a dose dependent manner, indicating that high energy UV light accelerates platelet glycolytic flux. Accumulation of lactic acid attributes to a decrease in plasma pH during storage. Since fresh plasma has buffering capacity, the pH would not be expected to have the same degree of correlation with in vivo recovery as lactic acid production does. Our linear regression analysis confirmed this. Interestingly glucose consumption, an upstream precursor for lactate production, demonstrated a relatively lower correlation coefficient with in vivo recovery than lactate production rate with no statistical significance (p>0.05). One possible explanation for this observation is that glucose consumption may not be completely linked to the glycolytic pathway leading exclusively to the end product of glycolysis, lactate. Indeed, many of the glucose-derived intermediates in glycolysis and the TCA cycle could also be transformed into fatty acids, lipids, amino acids and proteins. An alternative explanation is that residual levels of glucose present in products at the start of storage may alter rates of glucose consumption during storage. Since the rate of glycolysis is directly related to the concentration of glucose, it is possible that this mechanism may be at work in introducing additional variation in the response mechanism.

There were several reasons for choosing the citrate plasticized ELP bag, rather than the polyolefin Sengewald bag as the illumination bag for the Mirasol PRT platelet process. A study on the spectrum of UV transmission through the two bags showed that the ELP bag material yields a reduction in transmission of short wavelength UV light (See, FIG. 4). The reduction effect of short wavelength UV light is very critical in order to avoid severe damage to treated platelet organelles such as mitochondria which maintains part of bioenergy ATP supply for platelet viability and function. Indeed, independent studies confirmed that mitochondrial function and structural integrity was well preserved after platelets were treated with 6.2 J/mL in the ELP bag and stored for up to 7 days even though glycolytic flux was accelerated (manuscript in submission). Interestingly, the results from previous work also demonstrated that products treated in an ELP bag exhibit increased oxygen consumption during storage as evidenced by lower pO₂ values at day 5 in treated products compared to controls during storage. In contrast, results from the study conducted in polyolefin Sengewald bags did not show this effect (FIG. 8g). In essence, a fixed energy delivery in a polyolefin Sengewald container is not equivalent to that observed in an ELP bag (data not shown). These results suggest that oxidative metabolism in products treated in the polyolefin Sengewald bag was disrupted as a result of mitochondrial damage. Clearly, exposure levels observed for products treated in the polyolefin container (Sengewald bag) represent a worst-case scenario with regard to overall UV light dose exposure. In addition, illumination in the ELP bag also has the added benefit of avoiding subsequent transfer to an ELP bag for storage, simplifying the PRT treatment platelet process.

These effects also suggest a bimodal action of UV light. Lower light transmission in a short wavelength region and higher transmission of longer, less energetic wavelengths in an ELP bag may actually increase both glycolytic and mitochondrial activity in tandem, resulting in a balancing of actions with regard to pH and product stability during storage. With higher light dose at higher energy wavelengths (short wavelengths), an increase of glycolysis and reduction in oxidative metabolism may occur, resulting in lower pH values and poorer cell quality during storage. As this study demonstrates, the Mirasol PRT treatment corresponding to 6.2 J/mL UV dose delivered to products in an ELP bag yield in vivo recovery at 51±18%, a value within a normal range of products currently used in standard clinical practice.

It is also important to note that the in vivo viability prediction from the linear regression analysis of in vitro platelet quality for one platelet process and storage system may not necessarily extrapolate to other platelet process or treatment systems. Because the mechanism underlying the platelet storage lesion development is not fully understood and the responses of platelets to various treatment factors could be different, the predictions available from one system may not apply to other systems. For example, lactate production and pH, demonstrated to be the best predictors for in vivo recovery in this report, may not be useful in determining the recovery and survival of frozen or cold storage platelets. Nevertheless, it is interesting to note that information from platelet lactate production and pH also served as good predictors of untreated platelet recovery for products stored for 5 Days at room temperature under normal conditions (See Table 3).

These observations further demonstrate the utility of in vitro measures of platelet quality parameters for estimation of product performance in vivo. The value of in vitro measures has been questioned due to lack of apparent correlations in a number of settings. The work presented here demonstrates, however, that these measures can provide a valuable indicator of platelet performance in vivo and may serve as a means for guiding development work of new techniques and new handling methodologies. Once these correlations are established, they may also be able to serve as surrogates for more direct but complicated and difficult in vivo evaluations. In the case of each new treatment modality, it may be necessary to establish the most effective and predictive measurements for in vivo performance by obtaining direct correlations between these parameters and in vivo performance as described here. In the work presented here, the most effective predictors of in vivo recovery for UV treated samples under the conditions utilized were found to be sample pH and Lactic acid production rates. The utility of these measures was demonstrated by their ability to guide further development work which has defined the treatment conditions for a new pathogen reduction technology, Mirasol PRT.

TABLE 1
In vitro cell quality parameters of the platelets treated with various doses of
UV light and stored for 5 days

| | Control (n = 7) | | | Medium UV dose (n = 5) | | |
|---|---|---|---|---|---|---|
| | Day 0 | Day 3 | Day 5 | Day 0 | Day 3 | Day 5 |
| Total cell # (10¹¹) | 3.52 +/− 0.31 | | | 2.93 +/− 0.44 | | |
| Plt count (10³/ul) | 1409 +/− 122 | 1383 +/− 142 | 1442 +/− 144 | 1052 +/− 159 | 1057 +/− 127 | 1113 +/− 157 |
| Lactate (mM) | 2.03 +/− 0.33 | 7.00 +/− 1.32 | 9.81 +/− 2.39 | 2.18 +/− 0.55 | 10.48 +/− 2.83 | 16.44 +/− 3.20 |
| Glucose (mM) | 19.09 +/− 0.68 | 17.17 +/− 1.33 | 15.09 +/− 1.56 | 18.06 +/− 1.26 | 14.42 +/− 2.01 | 10.86 +/− 2.75 |
| pH @22° C. | 7.30 +/− 0.04 | 7.47 +/− 0.08 | 7.37 +/− 0.10 | 7.29 +/− 0.03 | 7.24 +/− 0.14 | 6.96 +/− 0.28 |
| pO2 (mmHg) | 81 +/− 47 | 81 +/− 27 | 62 +/− 11 | 60 +/− 15 | 82 +/− 24 | 68 +/− 17 |
| pCO2 (mmHg) | 63 +/− 6 | 29 +/− 2 | 29 +/− 2 | 56 +/− 2 | 28 +/− 4 | 25 +/− 3 |
| P-selectin (%) | 0 | 13 +/− 6 | 16 +/− 10 | 0 | 41 +/− 6 | 52 +/− 8 |
| HSR (%) | 85 +/− 8 | 85 +/− 7 | 84 +/− 8 | 86 +/− 3 | 78 +/− 6 | 70 +/− 7 |
| Swirl | 3.0 +/− 0.0 | 2.7 +/− 0.8 | 2.7 +/− 0.8 | 3.0 +/− 0.0 | 3.0 +/− 0.0 | 2.6 +/− 0.5 |
| Lactate rate | | 0.045 +/− 0.011 | | | 0.104 +/− 0.041 | |
| (mmol/10¹² plt/h) | | | | | | |
| Glucose rate | | 0.024 +/− 0.010 | | | 0.0618 +/− 0.015 | |
| (mmol/10¹² plt/h) | | | | | | |

| | High UV dose (n = 6) | | |
|---|---|---|---|
| | Day 0 | Day 3 | Day 5 |
| Total cell # (10¹¹) | 3.10 +/− 0.16 | | |
| Plt count (10³/ul) | 1114 +/− 58 | 1069 +/− 56 | 1032 +/− 175 |
| Lactate (mM) | 2.48 +/− 1.22 | 16.97 +/− 2.59 | 24.91 +/− 3.82 |
| Glucose (mM) | 17.23 +/− 1.10 | 11.27 +/− 2.13 | 6.22 +/− 3.15 |
| pH @22° C. | 7.33 +/− 0.03 | 7.08 +/− 0.10 | 6.53 +/− 0.23 |
| pO2 (mmHg) | 95 +/− 19 | 83 +/− 22 | 88 +/− 30 |
| pCO2 (mmHg) | 51 +/− 5 | 33 +/− 3 | 20 +/− 4 |
| P-selectin (%) | 0 | 57 +/− 10 | 71 +/− 11 |
| HSR (%) | 88 +/− 5 | 70 +/− 7 | 56 +/− 24 |
| Swirl | 3.0 +/− 0.0 | 2.0 +/− 0.0 | 1.2 +/− 0.4 |
| Lactate rate | | 0.186 +/− 0.033 | |
| (mmol/10¹² plt/h) | | | |
| Glucose rate | | 0.075 +/− 0.022 | |
| (mmol/10¹² plt/h) | | | |

TABLE 2
Correlation of in vitro cell quality and in vivo platelet recovery

| | | Correlation | | |
|---|---|---|---|---|
| | | efficient | | |
| | Regression equation | (r value) | F value | P value |
| Lactate rate | y = −372.08x + 75.964 | 0.909 | 79.83 | 0.007 |
| pH | y = 50.041x − 312.4 | 0.8831 | 64.74 | 0.031 |
| Glucose rate | y = −710.32x + 73.359 | 0.8398 | 58.39 | NS |
| p-Selectin | y = −0.6615x + 72.57 | 0.839 | 53.43 | NS |
| HSR | y = 0.8167x − 21.145 | 0.6492 | 33.05 | NS |
| Swirl | y = 16.701x + 0.4253 | 0.6586 | 20.62 | NS |
| pCO₂ | y = 3.1956x − 42.924 | 0.6432 | 17.03 | NS |
| pO₂ | y = −0.4286x + 67.732 | 0.4094 | 7.16 | NS |

*NS means not significant when p value ≧0.05

TABLE 3
Predicted recoveries from lactate production rate and pH parameters and
measured recovery for control and test platelets treated with Mirasol PRT.

| | Lactate prediction | | pH prediction | | Measured |
|---|---|---|---|---|---|
| | Lactate rate | Recovery (%) | pH at 22° C. | Recovery (%) | Recovery* |
| Mirasol PRT (n = 30) | 0.056 +/− 0.012 | 55.1 +/− 11.8 | 7.13 +/− 0.13 | 44.4 +/− 1.1 | 51.4 +/− 18.6 (n = 24) |
| Control (n = 20) | 0.032 +/− 0.006 | 64.1 +/− 12.0 | 7.48 +/− 0.06 | 61.9 +/− 0.4 | 67.8 +/− 13.4 (n = 22) |

*The data is published by AuBuchon et al. (AuBuchon JP, Herschel L, Roger J et al. Efficacy of apheresis platelets treated with riboflavin and ultraviolet light for pathogen reduction. Transfusion 2004; 44: 16A.)

### EXAMPLE 2

### Permeability of Citrate Plasticized Poly(Vinyl Chloride) Containers After Exposure to Ultraviolet Light

1. Introduction

The permeability of citrate plasticized poly(vinyl chloride) containers with respect to O₂ and CO₂ was characterized before and after exposure to electromagnetic radiation to verify their usefulness in the present methods. It is a goal of the present invention to provide containers that exhibit a permeability with respect to O₂ and CO₂ that does not decrease significantly upon exposure to electromagnetic radiation having wavelengths, radiant energies and radiant powers useful for processing blood and blood components. Further, it is a goal of the present invention to provide multifunctional containers useful for both storing and treating blood and blood components with electromagnetic radiation so as to avoid unnecessary and resource intensive additional sample transfer steps.

For platelet viability, platelets must be stored in a material that allows transmission of O₂ and CO₂, which are elements of platelet aerobic metabolism. In one embodiment of the present methods, pathogens in platelet containing samples are reduced by exposure to ultraviolet electromagnetic radiation. It is, therefore, beneficial to use a sample container in these methods that allows transmission of O₂ and CO₂ and does not exhibit significant decrease in gas permeability characteristics after exposure to ultraviolet electromagnetic radiation. In the present studies, transmission rates of O₂ and CO₂ were measured for citrate plasticized poly(vinyl chloride) ELP platelet storage bags (38% weight percent of n-butyryltri-n-hexyl citrate) that were systematically exposed to a selected net radiant energies useful for treatment of platelet-containing samples. Because the bag material must be dry and free from blood products for the gas permeability testing, saline with riboflavin are used to simulate actual use conditions during illumination.

2. Experimental

In the present study, 1 liter citrate plasticized poly(vinyl chloride) ELP platelet storage bags are filled with 250 mL of saline (to simulate the platelet product volume) and 28 mL of riboflavin. The bags are placed in an illuminator and exposed to UV electromagnetic radiation. The bags are removed after a target energy equal to 0 (control sample) or 5 J/cm² (test sample) is delivered. The fluid is subsequently removed, the bags are cut open, and the insides are blotted dried. Three replicates of test articles are performed at two energy points. Six of the test articles are used for O₂ transmission testing and the remaining six test articles are used for CO₂ transmission testing. Gas transmission testing is performed according to ASTM D3985 modified for 90% RH and CO₂ using established protocols. Tables 4 and 5 provide the test article matrix and a summary of illumination conditions, respectively, for the present study

TABLE 4
Test Article Matrix

| | 0 J/cm² | |
|---|---|---|
| | (Controls) | 5 J/cm² |
| O₂ | 3 bags (single | 3 bags (single |
| Transmission | side) | side) |
| CO₂ | 3 bags (single | 3 bags (single |
| Transmission | side) | side) |

TABLE 5
Summary of Illumination Conditions

| Test Consideration | Value |
|---|---|
| Light Wavelength Spectrum | Broadband UV |
| Illuminator Configuration | P/N 777074-510, 12 |
| | lamp UV 110 V, 60 Hz |
| Light Intensity (J/cm² · min) | P/N 777074-563 |
| Light Energy (J/cm²) | 0, 5 |
| Light Exposure Time | TBD by Illuminator |
| | Mapping |
| Solution Aspect Ratio (thickness) | 0.80 cm |
| Temperature | 30° C. ± 2° C. |
| Mixing Technique | “Linear” |
| Mixing Speed | 120 ± 5 cpm |
| Illumination Bag Type | 1L ELP without label |
| Volume of saline | 250 ml |
| Volume of 500 μM Riboflavin | 28 ml |
| Final bag volume | 278 ml |

The following procedure is adopted for evaluation of CO₂ and O₂ transmission rates of citrate plasticized poly(vinyl chloride) ELP platelet storage bag exposed to ultraviolet electromagnetic radiation.
1. Obtain 12 EtO sterilized 1 L ELP Illumination Bags.
2. Record part number and lot number on Data Collection Sheet.
3. Label bags with O₂ or CO₂ and the target energy (i.e., O₂ 5 J/cm²).
4. Fill the bag with 250 ml of sterile saline and 28 ml of 500 μM riboflavin solution. Record the saline and riboflavin lot numbers on the Data Collection Sheet.
5. For the controls (0 J/cm²), fill the bag with saline and riboflavin solution then empty it and proceed to Step 15.
6. Each test article is illuminated to 5.0 J/cm² per the OAI UV Powermeter light mapping in the Illuminator Mapping Function Verification (PIN 777074-563). This takes approximately 8½ minutes.
7. Set up the Illuminator through the Query screen. Verify that the illuminator is configured to run in EXPOSURE mode with 320nm lights, the temperature set point (SET TEMP) is 30° C., and the ENDPOINT is set to 5.0 J/cm². Verify that the agitation rate is set to 120 cpm. Record that the illuminator is configured for exposure mode in the Data Collection Sheet.
8. Ensure that the Lamp Mapping Function Verification (P/N 777074-563) has been completed prior to treating any products.
9. Secure the fluid-filled bag on the illuminator platen.
10. Measure the initial test article temperature (° C.) with an IR thermometer and record.
11. At the beginning of each illumination process, verify within the first 30 seconds of operation that all three “All is Well” indicator lights have come on. Circle Yes/No on the Data Collection Sheet when verified. If the lights don't come on, stop the process until the problem can be remedied.
12. Illuminate each test article to deliver a total energy dose of 5.0 J/cm².
13. Record the temperature by IR thermometer at the end of each illumination procedure.
14. After illumination, remove the bag from the illuminator.
15. Empty the illumination bag of fluid.
16. Cut the bottom of the bag off and blot dry the inside of the bag with a Kimwipe.
17. Place each set of bags in a Proper Sterilization pouch labeled with the appropriate test condition.
18. Test articles with respect to O₂ and CO₂ transmission.
   3. Data and Data Analysis

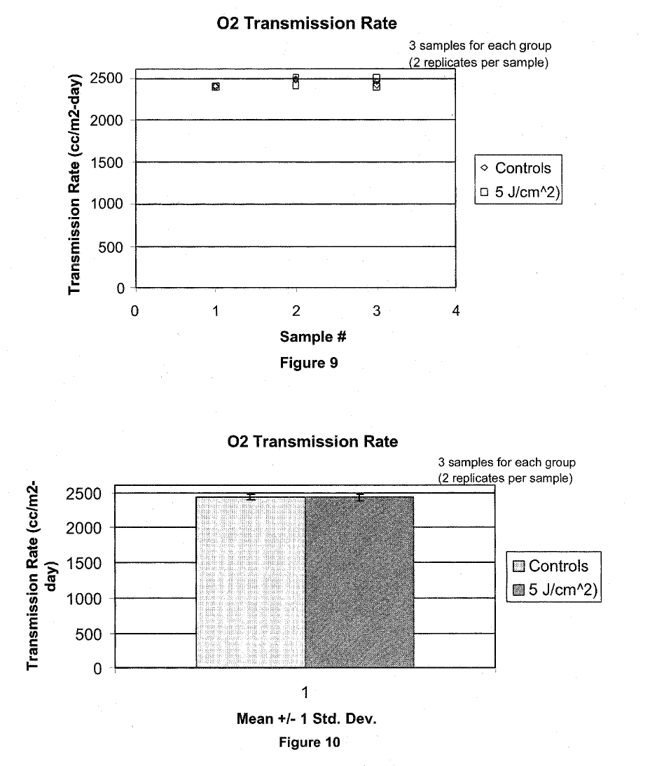

Table 6 lists the results and summary statistics for the O₂ transmission rate, including calculated mean and standard deviation. Additionally, a t-test (alpha=0.05) of the means was performed for each group of test and control samples. FIG. 9 shows measured O₂ transmission rates for each sample (three bag samples for each group, two replicates per sample). FIG. 10 shows the mean for each group (test and control) with error bars indicating ±1 standard deviation. As shown in FIG. 10, the measured mean values of O₂ transmission rates for test and control experiments are within respective standard deviations. In addition, the determined means of O₂ transmission rates are not significantly different between test and control samples per the t-test evaluation.

TABLE 6
Measured O₂ Transmission Rates

| | | O2 Transmission Rate | | | |
|---|---|---|---|---|---|
| | | (cc/m 2-day) | (cc/m 2-day) | (cc/100 in 2-day) | (cc/100 in 2-day) |
| Sample # | Replicate # | Controls | 5 J/cm\hat{ }2) | Controls | 5 J/cm\hat{ }2 |
| 1 | 1 | 2397 | 2404 | 503 | 505 |
| 1 | 2 | 2404 | 2407 | 505 | 506 |
| 2 | 1 | 2489 | 2507 | 523 | 502 |
| 2 | 2 | 2466 | 2415 | 518 | 507 |
| 3 | 1 | 2418 | 2390 | 508 | 526 |
| 3 | 2 | 2458 | 2497 | 516 | 524 |
| Mean | | 2439 | 2437 | 512 | 512 |
| Std Dev. | | 37 | 51 | 8 | 10 |
| n | | 6 | 6 | 6 | 6 |

| | | |
|---|---|---|
| t-test of means, | not sig. different | not sig. different |
| alph = .05 | | |

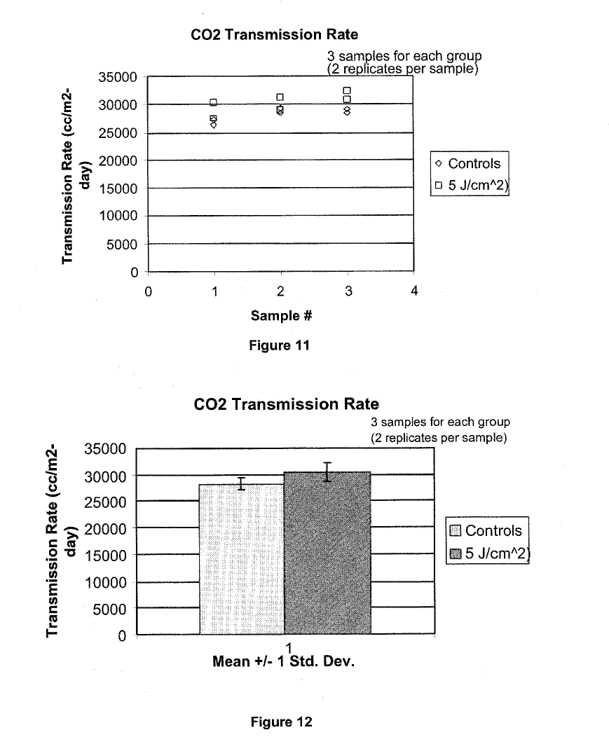

Table 7 lists the results and summary statistics for the CO₂ transmission rate, including calculated mean and standard deviation. Additionally, a t-test (alpha=0.05) of the means was performed for each group of test and control samples. FIG. 11 shows measured CO₂ transmission rates for each sample (three bag samples for each group, two replicates per sample). FIG. 12 shows the mean of CO₂ transmission rates for each group (test and control) with error bars indicating ±1 standard deviation. As shown in Table 7, FIG. 11 and FIG. 12, a statistically significant change in the rate of COtransmission is observed upon exposure to ultraviolet radiation. Although this increase is statistically significant, the CO₂ transmission rates increases slightly 9about 8%) upon exposure to ultraviolet radiation, as opposed to decreasing. Further, the magnitude of the observed increase is not enough to impact platelet quality or viability, and thus, is not expected to have clinical significance.

TABLE 7
Measured CO₂ Transmission Rates

| | | CO2 Transmission Rate | | | |
|---|---|---|---|---|---|
| | | (cc/m 2-day) | (cc/m 2-day) | (cc/100 in 2-day) | (cc/100 in 2-day) |
| Sample # | Replicate # | Controls | 5 J/cm\hat{ }2) | Controls | 5 J/cm\hat{ }2 |
| 1 | 1 | 26396 | 30321 | 1703 | 1956 |
| 1 | 2 | 27230 | 27570 | 1757 | 1779 |
| 2 | 1 | 29311 | 28932 | 1891 | 1867 |
| 2 | 2 | 28544 | 31285 | 1842 | 2018 |
| 3 | 1 | 28394 | 30953 | 1832 | 1997 |
| 3 | 2 | 29253 | 32586 | 1887 | 2102 |
| Mean | | 28188 | 30275 | 1819 | 1953 |
| Std Dev. | | 1157 | 1786 | 75 | 115 |
| n | | 6 | 6 | 6 | 6 |

| | | |
|---|---|---|
| t-test of means, | sig. different | sig. different |
| alph = .05 | | |

### STATEMENTS REGARDING INCORPORATION BY REFERENCE AND VARIATIONS

All references throughout this application, for example patent documents including issued or granted patents or equivalents; patent application publications; unpublished patent applications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Separate embodiments of the invention are also intended to be encompassed wherein the terms “comprising” or “comprise(s)” or “comprised” are optionally replaced with the terms, analogous in grammar, e.g.; “consisting/consist(s)” or “consisting essentially of/consist(s) essentially of” to thereby describe further embodiments that are not necessarily coextensive.

The invention has been described with reference to various specific and preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all subranges and individual values are intended to be encompassed as if separately set forth. This invention is not to be limited by the embodiments disclosed, including any shown in the drawings or exemplified in the specification, which are given by way of example or illustration and not of limitation. The scope of the invention shall be limited only by the claims.

We claim:

1. A method for reducing pathogens in a biological sample; said method comprising the steps of:
providing a container holding said biological sample; wherein said container comprises a polymeric material and a citrate plasticizer, wherein said container transmits electromagnetic radiation having a distribution of wavelengths; and
exposing said container to electromagnetic radiation, wherein electromagnetic radiation having said distribution of wavelengths is transmitted by said container and is at least partially absorbed by said biological sample, thereby reducing said pathogens in the biological sample;
wherein the transmission of electromagnetic radiation having said distribution of wavelengths by said container is substantially constant during exposure to electromagnetic radiation.

2. The method of claim 1 wherein the transmission of electromagnetic radiation having said distribution of wavelengths by said container is constant to within 10% during exposure to electromagnetic radiation.

3. The method of claim 1 wherein the transmission of electromagnetic radiation having said distribution of wavelengths by said container is constant to within 5% during exposure to electromagnetic radiation.

4. The method of claim 1 wherein said distribution of wavelengths is in the ultraviolet region of the electromagnetic spectrum, visible region of the electromagnetic spectrum or both.

5. The method of claim 1 wherein said container is exposed to said electromagnetic radiation for a time period selected from the range of about 0.1 minutes to about 30 minutes.

6. The method of claim 1 wherein said container is exposed to a net radiant energy selected from the range of about 0.1 Joules cm⁻² to about 10 Joules cm⁻².

7. The method of claim 1 wherein said electromagnetic radiation having said distribution of wavelengths has wavelengths selected over the range of about 285 nanometers to about 365 nanometers.

8. The method of claim 1 wherein said citrate plasticizer is selected from the group consisting of:
triethyl citrate;
acetyltriethyl citrate;
n-butyryltri-n-hexyl citrate; and
acetyltri-n-butyl citrate.

9. The method of claim 1 wherein said container further comprises at least one additional citrate plasticizer.

10. The method of claim 1 wherein the concentration of said citrate plasticizer is selected over the range of about 25% to about 50% by weight.

11. The method of claim 1 wherein the concentration of said citrate plasticizer is about 38% by weight.

12. The method of claim 1 wherein said polymeric material is poly(vinyl chloride).

13. The method of claim 1 wherein said container further comprises an additional additive is selected from the group consisting of: a plasticizer; a light stabilizer; a heat stabilizer; an antioxidant; a flame retardant; a mold release agent; and a nucleating agent;

14. The method of claim 1 wherein said biological sample is a blood component.

15. The method of claim 1 wherein said biological sample is a fluid.

16. The method of claim 14 wherein said blood component is selected from the group consisting of: platelets; plasma; red blood cells; white blood cells; and plasma proteins.

17. The method of claim 14 wherein said biological sample further comprises a photosensitizer.

18. The method of claim 17 wherein said photosensitizer is 7,8-dimethyl-10-ribityl isoalloxazine.

19. The method of claim 1 wherein said biological sample comprises a material selected from the group consisting of: whole blood; a blood component; a red blood cell-containing blood component; a plasma-containing blood component; a platelet-containing blood component; a white blood cell-containing blood component; a solution containing one or more proteins derived from blood; and a peritoneal solution.

20. A method for reducing pathogens in a biological sample; said method comprising the steps of:
providing a container holding said biological sample; wherein said container comprises poly(vinyl chloride) and at least one citrate plasticizer, wherein said container transmits electromagnetic radiation having a selected distribution of wavelengths and wherein the transmission of electromagnetic radiation having said selected distribution of wavelengths by said container is substantially constant during exposure to electromagnetic radiation;
measuring the percentages of transmission of said container as a function of wavelength over said selected distribution of wavelengths;
generating electromagnetic radiation using a source of electromagnetic radiation;
monitoring the power of said electromagnetic radiation generated by said light source;
calculating a radiant power delivered to said biological sample using said measured percentages of transmission of said container;
determining an exposure time of said biological sample required to provide a desired extent of pathogen reduction; and
exposing said container to electromagnetic radiation for said exposure time, wherein electromagnetic radiation having said selected distribution of wavelengths is transmitted by said container and is at least partially absorbed by said biological sample, thereby reducing said pathogens in the biological sample.

21. The method of claim 20 wherein said citrate plasticizer is selected from the group consisting of:
triethyl citrate;
acetyltriethyl citrate;
n-butyryltri-n-hexyl citrate; and
acetyltri-n-butyl citrate.

22. The method of claim 20 wherein the concentration of said citrate plasticizer is selected over the range of about 25% to about 50% by weight.

23. The method of claim 20 wherein the concentration of said citrate plasticizer is about 38% by weight.

24. The method of claim 1 wherein said polymeric material is poly(vinyl chloride).

25. A method for reducing pathogens in a biological sample; said method comprising the steps of:
providing a container holding said biological sample; wherein said container comprises a polymer and at least one optical filtering additive, wherein the composition and concentration of said additive is selected so that electromagnetic radiation having a first distribution of wavelengths is at least partially transmitted by said container and transmission of electromagnetic radiation having a second distribution of wavelengths is substantially prevented, wherein electromagnetic radiation having said first distribution of wavelengths is capable of initiating pathogen reduction of said biological sample; and
exposing said container to electromagnetic radiation, wherein transmission of electromagnetic radiation of said second distribution of wavelengths is substantially prevented, and wherein electromagnetic radiation having said first distribution of wavelengths is transmitted by said container and is at least partially absorbed by said biological sample, thereby reducing said pathogens in the biological sample.

26. The method of claim 25 wherein said additive is immobilized within a polymer network of said polymer.

27. The method of claim 25 wherein said additive and said polymer are copolymers, wherein said additive is covalently bonded to said polymer.

28. The method of claim 25 wherein said additive is selected from the group consisting of one or more amino acids, one or more proteins, one or more peptides, one or more nucleic acids and one or more oligonucleotides.

29. The method of claim 25 wherein said additive is one or more citrate plasticizer selected from the group consisting of: triethyl citrate; acetyltriethyl citrate; n-butyryltri-n-hexyl citrate; and acetyltri-n-butyl citrate.

30. The method of claim 25 wherein said additive is one or more amino acids selected from the group consisting of tyrosine, histidine, phenylalanine and tryptophan.

31. The method of claim 25 wherein said polymer is poly(vinyl chloride).

32. The method of claim 25 wherein said electromagnetic radiation having said second distribution of wavelengths is capable of damaging at least one component of said biological sample
